(19) United States
(12) Patent Application Publication
(10) Pub. No.: US 2022/0348569 A1
(43) Pub. Date: Nov. 3, 2022

(54) RESPIRATORY VIRUS INHIBITORS

(71) Applicant: New York Blood Center, Inc., New York, NY (US)

(72) Inventors: Asim Kumar Debnath, Fort Lee, NJ (US); Francesca Curreli, West New York, NY (US); Christopher D. Hillyer, New York, NY (US); Shahad Ahmed, Fresh Meadows, NY (US)

(21) Appl. No.: 17/565,445

(22) Filed: Dec. 29, 2021

Related U.S. Application Data
(60) Provisional application No. 63/178,446, filed on Apr. 22, 2021.
(60) Provisional application No. 63/278,965, filed on Nov. 12, 2021.
(60) Provisional application No. 63/264,698, filed on Nov. 30, 2021.
(60) Provisional application No. 63/264,740, filed on Dec. 1, 2021.

(51) Int. Cl. C07D 417/06; A61P 31/12

(52) U.S. Cl. CPC C07D 417/06; A61P 31/12

(57) ABSTRACT

Disclosed herein are compounds which demonstrate activity against respiratory viruses, such as coronaviruses.

### CROSS REFERENCE TO RELATED APPLICATIONS

This application claims the benefit of U.S. Provisional patent applications 63/178,446 filed Apr. 22, 2021, 63/278,965 filed Nov. 12, 2021, 63/264,698, filed Nov. 30, 2021, and 63/264,740, filed Dec. 1, 2021, the entire contents of all of which are incorporated by reference herein.

### FIELD

Methods of treating respiratory viruses, such as coronaviruses, are described herein.

### BACKGROUND

The outbreak of a coronavirus disease 2019 (COVID-19), caused by a novel coronavirus, SARS-CoV-2, was first reported in December 2019 in Wuhan, China. In March 2020, the World Health Organization (WHO) declared this outbreak a global pandemic. Coronaviruses (CoVs) belong to the taxonomical family of Coronaviridae and are positive-sense, enveloped, single-stranded RNA viruses. Two other related coronaviruses that caused major outbreaks in recent years are severe acute respiratory syndrome coronavirus (SARS-CoV) in 2003 (originated in China) and Middle East respiratory syndrome coronavirus (MERS-CoV) in 2012 (originated in Saudi Arabia). Based on the CDC report, 8,098 people were infected in 26 countries, and that 774 died from SARS-CoV, whereas a 2020 update from WHO reported 2566 confirmed cases with 882 death from MERS-CoV since 2012 (http://www.emro.who.int/health-topics/mers-cov/mers-outbreaks.html). Based on data from Johns Hopkins Coronavirus Resource center, as of Dec. 1, 2021, more than 263 million cases with more than five million deaths were reported globally. In the United States, over 48 million cases and more than 780,000 deaths have been reported.

Worldwide, several other COVID-19 vaccines have been approved and deployed. Despite this breakthrough development, vaccines may not reach all individuals worldwide. Besides, vaccine hesitancy is also occurring and expected to continue to play a major role in getting an entire population vaccinated in any country. Furthermore, “breakthrough” cases of COVID-19 infection have been reported among people who were fully vaccinated.

Although the US FDA approved several repurposed drugs such as remdesivir and lopinavir/ritonavir for emergency use, they have shown limited usefulness in clinical settings. The US FDA also approved several monoclonal antibody-based therapies, but their cost and accessibility may be prohibitive to a significant part of the world. Furthermore, most of the approved vaccines and antibody-based therapies show substantial loss of potency against several SARS-CoV-2 variants recently identified in the UK (Alpha variant; B.1.1.7), South Africa (Beta variant; B.1.351, and Brazil (Gamma variant; P.1). Recently, the Delta (B.1.617.2, identified initially in India) and the Omicron (B.1.1.529) variants are spreading rapidly. While vaccines are critical in preventing infection and severe illness, therapeutic drugs play a crucial role in combatting the disease in individuals who get infected. While vaccines are critical in preventing people from getting the viruses and becoming seriously ill, therapeutic drugs are still critically important to have in the arsenal to combat the disease for those who get infected. Therapeutic drugs may help patients with COVID-19 infection to become seriously ill, avoid hospitalization, and death. Therefore, the development of highly potent novel drugs with pancoronavirus activity with minimal toxicity is urgently needed.

### SUMMARY

Disclosed herein are methods of treating a respiratory infection comprising administering a compound having a structure represented by Formula (I) :

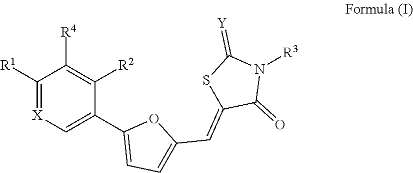

or a pharmaceutically acceptable salt thereof, wherein X is CH or N; Y is S, O, or NH; R¹ is H, halogen (for example Cl, F, or Br), or C₁₋₆ alkyl; R² is H, halogen (for example Cl, F, or Br), or C₁₋₆ alkyl; and R³ is H, methyl, ethyl, isobutyl, methoxyethyl, phenylethyl, allyl, propynyl, cyclohexyl, C₁₋₆ alkyl, C₁₋₆ alkoxyalkyl, C₁₋₆ alkenyl, C₁₋₆ alkynyl.

Also disclosed herein are methods of preventing a respiratory infection comprising administering a compound having a structure represented by Formula (I):

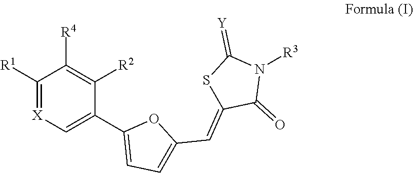

or a pharmaceutically acceptable salt thereof, wherein X is CH or N; Y is S, O, or NH; R¹ is H, halogen (for example Cl, F, or Br), or C₁₋₆ alkyl; R² is H, halogen (for example Cl, F, or Br), or C₁₋₆ alkyl; and R³ is H, methyl, ethyl, isobutyl, methoxyethyl, phenylethyl, allyl, propynyl, cyclohexyl, C₁₋₆ alkyl, C₁₋₆ alkoxyalkyl, C₁₋₆ alkenyl, C₁₋₆ alkynyl; wherein the method comprises administering the compound, or a pharmaceutical composition comprising the compound, to a subject exposed to, or suspecting of being exposed to, the respiratory virus.

In some embodiments, the compound has the structure of Formula (II):

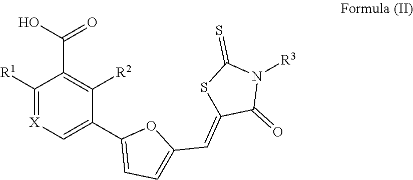

or a pharmaceutically acceptable salt thereof, wherein X is CH or N; R¹ is H, halogen (for example Cl, F, or Br), or C₁₋₆ alkyl; R² is H, halogen (for example Cl, F, or Br), or C₁₋₆ alkyl; and R³ is H, C₁₋₆ alkyl, C₁₋₆ alkoxyalkyl, C₁₋₆ alkenyl, C₁₋₆ alkynyl, C_{3- 6} cycloalkyl, —(C₁₋₄ alkyl)-phenyl.

In some embodiments, the compound has a structure of Formula (III):

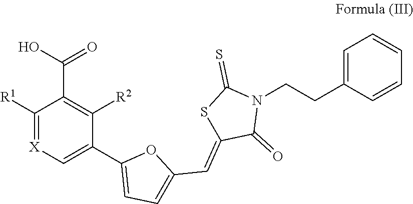

or a pharmaceutically acceptable salt thereof, wherein X is CH or N; R¹ is H, halogen (for example Cl, F, or Br), or C₁₋₆ alkyl; and R² is H, halogen (for example Cl, F, or Br), or C₁₋₆ alkyl.

In some embodiments, the compound has a structure of Formula (IV):

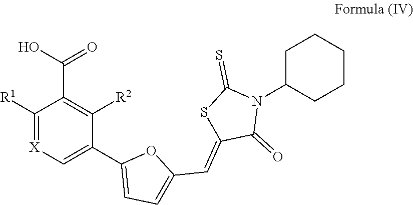

or a pharmaceutically acceptable salt thereof, wherein X is CH or N; R¹ is H, halogen (for example Cl, F, or Br), or C₁₋₆ alkyl; and R² is H, halogen (for example Cl, F, or Br), or C₁₋₆ alkyl.

In some embodiments, R² is H. In some embodiments, R¹ is methyl, Cl, or Br. In some embodiments, R¹ is H. In some embodiments, R³ is phenylethyl or cyclohexyl.

In some embodiments, the compound is selected from the compounds of Table 1. In some embodiments, the compound is

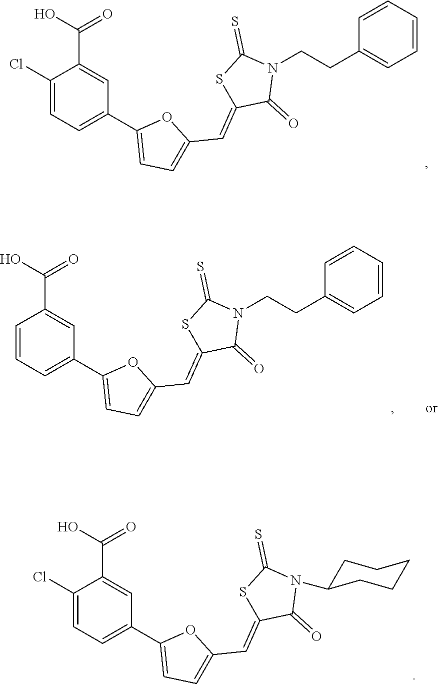 [SMILES: O=C(O)c1cc(-c2ccc(/C=C3\SC(=S)N(CCc4ccccc4)C3=O)o2)ccc1Cl ; O=C(O)c1cccc(-c2ccc(/C=C3\SC(=S)N(CCc4ccccc4)C3=O)o2)c1 ; O=C(O)c1cc(-c2ccc(/C=C3\SC(=S)N(C4CCCCC4)C3=O)o2)ccc1Cl]

In some embodiments, the subject in need thereof has the respiratory infection confirmed by detection of a respiratory virus in a biological sample from the subject.

In some embodiments, the respiratory virus is coronavirus. In some embodiments, the compound is a pancoronavirus inhibitor. In some embodiments, the coronavirus is MERS-CoV. In some embodiments, the coronavirus is SARS-CoV. In some embodiments, the coronavirus is SARS-CoV-2.

In some embodiments, the compound is administered orally. In some embodiments, the compound is administered intranasally.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1A-E. Validation of the SARS-CoV-2, SARS-CoV, and MERS-CoV pseudoviruses and ACE2 and DPP4 expression in different cell lines. FIG. 1A depicts an immunoblot to validate the incorporation of the S spike protein in the SARS-CoV and SARS-CoV-2 pseudoviruses using the SARS spike protein antibody (Novus Biologicals), which targets the spike protein S2. FIG. 1B depicts an immunoblot to validate the incorporation of the S spike protein in the MERS-CoV pseudovirus using a MERS-coronavirus spike protein S2 polyclonal antibody (Invitrogen). Infection of cells expressing different levels of ACE2 with the same amounts of SARS-CoV-2 pseudovirus (FIG. 1C), the same amounts of SARS-CoV pseudovirus (FIG. 1D), or infection of cells expressing different levels of DPP4, with the same amounts of MERS-CoV pseudovirus (FIG. 1E). Columns represent the means±standard deviations (n=4). FIG. 1F depicts an immunoblot of cell lysates to evaluate ACE2 expression and g) DPP4 expression. β-Actin was used as a loading control.

FIG. 2. SARS-CoV-2 mediated cell-to-cell fusion inhibition assay. Jukat cells expressing the SARS-CoV-2 full Spike wild-type gene from Wuhan-Hu-1 isolate and the luciferase gene were used as donor cells, and the 293T/ACE2 as acceptor cells. Jurkat cells were preincubated for 1 hr with different concentrations of the compounds of the present disclosure. Positive represent 293T/ACE2 cells cocultured with Jurkart cells in the absence of the compounds of the present disclosure. Jurkat-Luc represents 293T/ACE2 cells cocultured with Jurkat cells expressing the luciferase gene only, in the absence of compounds. Columns represent the means±standard deviations (n=2-4).

FIG. 3A-D depicts the evaluation of binding affinity of Compound 1 and Compound 2 to SARS-CoV-2 active trimmer and SARS-CoV-2 51 subdomain by SPR. Kinetics fitting curve (sensogram) of SARS-CoV-2 trimer to Compound 1 (FIG. 3A) and Compound 2 (FIG. 3B). Kinetics fitting curve (sensogram) of SARS-CoV-2 51 subdomain to Compound 1 (FIG. 3C) and Compound 2 (FIG. 3D). The binding affinity K_{D} and the kinetic parameters, kₒₙ and k_{off}, of Compound 1 and Compound 2 are depicted in Table 17.

### DETAILED DESCRIPTION

A series of compounds have been identified which demonstrate potent inhibition against respiratory viruses, including coronaviruses such as SARS-CoV, SARS-CoV-2, and MERS-CoV. Moreover, the compounds may inhibit laboratory synthesized mutants, mimicking the SARS-CoV variants including, but not limited to, B.1.1.7 UK (Alpha), B.1.351 RSA (Beta), B.1.617.2 India (Delta), and B.1.1.529 (Omicron). The compounds of the present disclosure demonstrate low nM IC₅₀ in a pseudovirus-based inhibition assay against coronaviruses. The compounds of the present disclosure also demonstrate complete inhibition of cytopathic effects (CPE) (IC₁₀₀ ) against a native live virus, SARS-CoV-2 (US_WA-1/2020) in Vero cells. The compounds of the present disclosure bind to the S2 subdomain of SARS-CoV-2 with higher avidity than the S1 subdomain of the spike protein. Subsequent cell-to-cell fusion assays confirmed that The compounds of the present disclosure efficiently prevent cell-to-cell fusion.

The life cycle of coronaviruses (CoVs), like all other enveloped viruses, starts with the virus's entry to the host cells initiated by the trimeric spike surface protein (S). The S protein is cleaved into S1 and S2 subunits in host cells by furin-like proteases. The S1 subunit of the spike protein attaches to the host cell surface receptor through its receptor-binding domain (RBD). SARS-CoV and SARS-CoV-2 bind to the host cell receptor angiotensin-converting enzyme 2 (ACE2), whereas MERS-CoV binds to dipeptidyl peptidase 4 (DPP4, also termed CD26). After the virus attaches, the S2 subunit undergoes conformational changes to form a trimeric hairpin structure by heptad repeat 1 (HR1) and repeat 2 (HR2). This process brings the viral and host cell membranes together for virus-cell fusion, a critical step for virus entry into host cells. It has been demonstrated that the spike protein of coronaviruses, including MERS-CoV, use a similar structural mechanism to fuse with the cell membranes as that of other class I membrane fusion proteins such as influenza virus, human immunodeficiency virus (HIV), and Ebola virus. However, some distinctions exist, such as its larger size, double cleavage site, and long six-helix bundle.

Due to the spike protein's surface exposure, it is the primary target for neutralizing antibodies and vaccines. Both S1, primarily the RBD domain, and S2 proteins, especially the HR1 domain, have been targeted for identifying and designing novel drugs. However, the RBD of the S1 domain of the spike proteins among various coronaviruses is less conserved. Therefore, antibodies that neutralized SARS-CoV effectively showed poor-cross-reactivity with SARS-CoV-2. Furthermore, several mutations have been reported to the RBD domain of SARS-CoV-2. Some of these mutations reduced the efficacy of antibodies and currently available vaccines. Therefore, the RBD may not be an ideal target for identifying broad spectrum viral inhibitors.

However, membrane fusion domains located in the S2 are mostly conserved and can be an ideal target for small molecule and peptide-based viral inhibitors. Therefore, drugs that target most conserved sites among coronaviruses will provide better broad-spectrum (such as pancoronavirus) antiviral activity. These drugs will be critically important to deal with future pandemics that are expected to emerge.

Thus, disclosed herein are 3-(5((4-oxo-3-phenethyl-2-thioxothiazolidin-5-ylidene)methyl)furan-2-yl)benzoic acid-based compounds that present highly potent activity against respiratory viruses, such as coronaviruses. Activity has been demonstrated against several coronaviruses including SARS-CoV (IC₅₀: as low as 13 nM), SARS-CoV-2 (IC₅₀: as low as 23 nM), and MERS-CoV (IC₅₀: as low as 76 nM) in pseudovirus-based assays with excellent selectivity indices (SI: as high as >5000) demonstrating the pancoronavirus inhibition of the compounds of the present disclosure. In some embodiments, compounds of the present disclosure can show 100% inhibition of CPE (IC₁₀₀) at 1.25 μM against SARS-CoV-2 (strain US_WA-1/2020). Furthermore, the most active compounds can also potently inhibited SARS-CoV-2 variants including the Alpha (B.1.1.7), Beta (B.1.351), and Delta (B.1.617.2) variants. One of the potent compounds binds to the prefusion spike protein trimer of SARS-CoV-2 as detected by surface plasmon resonance (SPR). Additionally, 3-(5-((4-oxo-3-phenethyl-2-thioxothiazolidin-5-ylidene)methyl)furan-2-yl)benzoic acid-based compounds of the present disclosure inhibited virus-cell fusion. Furthermore, compounds of the present disclosure demonstrate drug-like properties determined by in vivo absorption, distribution, metabolism, and excretion (ADME) data. In vivo PK of the compounds in rats demonstrates excellent half-life (t_{1/2}) of 11.3 hr, mean resident time (MRT) of 14.2 hr, and oral bioavailability.

Herein, the present disclosure discloses a series of compounds that show highly potent activity against respiratory viruses, including the coronaviruses SARS-CoV, SARS-CoV-2, and MERS-CoV. Furthermore, the most active compounds also potently inhibit SARS-CoV-2 variants including, but not limited to the Alpha, Beta, and Delta variants.

In some embodiments, disclosed herein are methods of treating a respiratory infection comprising administering a compound having a structure represented by Formula (I):

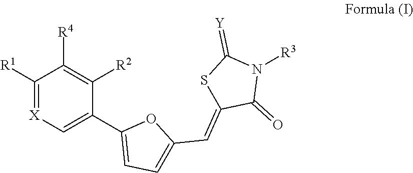

or a pharmaceutically acceptable salt thereof,

wherein X is CH or N;

Y is S, O, or NH;

R¹ is H, halogen, for example Cl, F, or Br, or C₁₋₆ alkyl;

R² is H, halogen, for example Cl, F, or Br, or C₁₋₆ alkyl; and

R³ is H, methyl, ethyl, isobutyl, methoxyethyl, phenylethyl, allyl, propynyl, cyclohexyl, C₁₋₆ alkyl, C₁₋₆ alkoxyalkyl, C₁₋₆ alkenyl, C₁₋₆ alkynyl.

In some embodiments, disclosed herein are methods of treating a respiratory infection comprising administering to a subject in need thereof a compound having a structure represented by Formula (II):

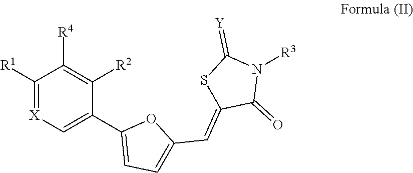

or a pharmaceutically acceptable salt thereof;

wherein X is CH or N;

Y is S, O, or NH;

R¹ is H, halogen, for example Cl, F, or Br, or C₁₋₆ alkyl and R⁴ is COOH, or R¹ is COOH or sulfonamido and R⁴ is H;

R² is H, halogen (for example Cl, F, or Br), or C₁₋₆ alkyl; and

R³ is H, C₁₋₆ alkyl, C₁₋₆ alkoxyalkyl, C₁₋₆ alkenyl, C₁₋₆ alkynyl, C₃₋₆ cycloalkyl, —(C₁₋₄ alkyl)-phenyl.

Also provided herein are methods for treating respiratory infections comprising administering to a subject in need thereof a compound having a structure represented by Formula (III):

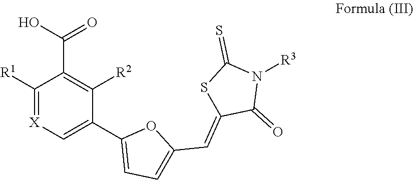

or a pharmaceutically acceptable salt thereof,

wherein

X is CH or N;

R¹ is H, halogen (for example Cl, F, or Br), or C₁₋₆ alkyl;

R² is H, halogen (for example Cl, F, or Br), or C₁₋₆ alkyl; and

R³ is H, C₁₋₆ alkyl, C₁₋₆ alkoxyalkyl, C₁₋₆ alkenyl, C₁₋₆ alkynyl, C₃₋₆ cycloalkyl, -(C₁₋₄ alkyl)-phenyl.

Also provided herein are methods for treating respiratory infections comprising administering to a subject in need thereof a compound having a structure represented by Formula (IV):

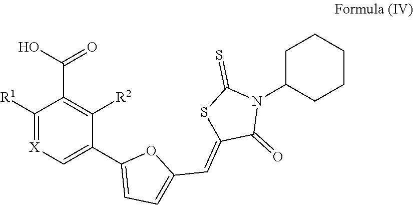

or a pharmaceutically acceptable salt thereof

wherein X is CH or N;

R¹ is H, halogen, for example Cl, F, or Br, or C₁₋₆ alkyl; and

R² is H, halogen, for example Cl, F, or Br, or C₁₋₆ alkyl.

Also provided herein are methods for reducing the likelihood of contracting a respiratory infections comprising administering to a person exposed to, or at increased risk of contracting, a respiratory virus, a compound having a structure represented by Formula II.

In some embodiments of the methods provided herein, R³ is phenylethyl or cyclohexyl. In some embodiments, R³ is methyl, ethyl, isobutyl, methoxyethyl, phenylethyl, allyl, propynyl, or cyclohexyl. In some embodiments of the formulae provided herein, R³ is phenylethyl. In some embodiments, R³ is cyclohexyl. In some embodiments, R² is H. In some embodiments, R¹ is methyl, Cl, or Br. In some embodiments, R¹ is H.

In some embodiments of the formulae provided herein, X is CH. In some embodiments, X is N.

In some embodiments of the formulae provided herein, R¹ is H. In some embodiments, R¹ is Cl. In some embodiments, R¹ is F. In some embodiments, R¹ is Br. In some embodiments, R¹ is methyl. In some embodiments, R¹ is C₁₋₂ alkyl. In some embodiments, R¹ is C₁₋₃ alkyl. In some embodiments, R¹ is C₁₋₆ alkyl. Some embodiments provided herein specifically include one or more of these alternatives while other embodiments specifically exclude one or more of these alternatives.

In some embodiments of the formulae provided herein, R² is H. In some embodiments, R² is Cl. In some embodiments, R² is F. In some embodiments, R² is Br. In some embodiments, R² is methyl. In some embodiments, R² is ethyl. In some embodiments, R² is C₁₋₂ alkyl. In some embodiments, R² is C₁₋₃ alkyl. In some embodiments, R² is C₁₋₆ alkyl. Some embodiments provided herein specifically include one or more of these alternatives while other embodiments specifically exclude one or more of these alternatives.

In some embodiments of the formulae provided herein, R³ is phenylethyl. In some embodiments, R³ is H. In some embodiments, R³ is methyl. In some embodiments, R³ is ethyl. In some embodiments, R³ is isobutyl. In some embodiments, R³ is methoxyethyl. In some embodiments, R³ is allyl, for example 3-allyl. In some embodiments, R³ is propynyl, for example, 2-propynyl. In some embodiments, R³ is cyclohexyl. In some embodiments, R³ is C₁₋₃ alkyl. In some embodiments, R³ is C₁₋₆ alkyl. In some embodiments, R³ is C₁₋₃ alkenyl. In some embodiments, R³ is C₁₋₆ alkenyl. In some embodiments, R³ is C₁₋₃ alkynyl. In some embodiments, R³ is C₁₋₆ alkynl. In some embodiments, R³ is C₁₋₃ alkoxyalkyl. In some embodiments, R³ is C₁₋₆ alkoxyalkyl. Some embodiments provided herein specifically include one or more of these alternatives while other embodiments specifically exclude one or more of these alternatives.

In some embodiments of the formulae provided herein, R⁴ is H when R¹ is COOH or sulfonamido. In some embodiments of the formulae provided herein, R⁴ is COOH, when R¹ is H, halogen (for example Cl, F, or Br), or C₁₋₆ (for example C₁₋₃ alkyl). Some embodiments provided herein specifically include one or more of these alternatives while other embodiments specifically exclude one or more of these alternatives.

Without being bound by theory, it is believed that the —COOH group of the compounds of the present disclosure interrupts the hairpin structure formation by interacting with the K547 and snuggly fitting to the hydrophobic groove created by three NHR regions. Further, such remarkable similarities in the mechanism of virus fusion and involvement of salt bridges in forming the 6-HB in HIV-1 gp41 and coronaviruses implies that this class of compounds can also interrupt the salt bridge formation by fitting in a cavity in the prefusion trimer of coronaviruses, thus preventing the 6-HB formation and eventually the fusion of viruses to host cells.

In some embodiments of the formulae provided herein, Y is S. In some embodiments, Y is O. In some embodiments, Y is NH. Some embodiments provided herein specifically include one or more of these alternatives while other embodiments specifically exclude one or more of these alternatives.

In further embodiments, the compound has the structure of any of the compounds in Table 1, or a pharmaceutically acceptable salt thereof. In some embodiments, the compound is selected from the group consisting of Compound 1, 2, 3, 4, or 18, or a pharmaceutically acceptable salt thereof. In some embodiments, the compound is a pharmaceutically acceptable salt of any one of the compounds illustrated in Table 1. The synthesis, purification, and analytical characterization of the compounds of the present disclosure may be performed analogously to procedures described in Katritzky et al., J Med Chem 52(23):7631-7639, 2009, and U.S. 2006/0287319, which are both incorporated by reference herein for all they disclose regarding synthesis, purification and analytical characterization of chemical compounds. In some embodiments, the compound is not Compound 5. In some embodiments, the compound is Compound 1, Compound 2, Compound 4, or Compound 18.

In some embodiments, the compound is selected from the compounds of Table 1, or a pharmaceutically acceptable salt thereof. In some embodiments, the compound is

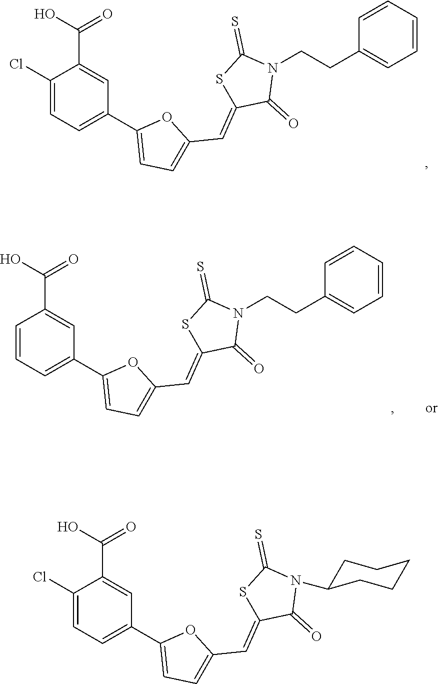 [SMILES: O=C(O)c1cc(-c2ccc(/C=C3\SC(=S)N(CCc4ccccc4)C3=O)o2)ccc1Cl ; O=C(O)c1cccc(-c2ccc(/C=C3\SC(=S)N(CCc4ccccc4)C3=O)o2)c1 ; O=C(O)c1cc(-c2ccc(/C=C3\SC(=S)N(C4CCCCC4)C3=O)o2)ccc1Cl]

or a pharmaceutically acceptable salt thereof.

Table 1 illustrates non-limiting examples of compounds for use in the methods provided herein.

TABLE 1

| Compound | Structure |
|---|---|
| 1 | 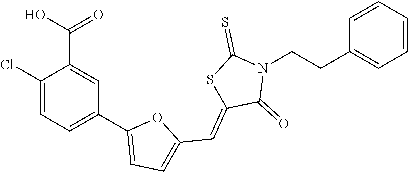 [SMILES: O=C(O)c1cc(-c2ccc(/C=C3\SC(=S)N(CCc4ccccc4)C3=O)o2)ccc1Cl] |
| 2 | 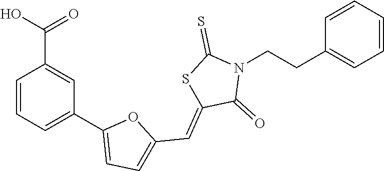 [SMILES: O=C(O)c1cccc(-c2ccc(/C=C3\SC(=S)N(CCc4ccccc4)C3=O)o2)c1] |
| 3 | 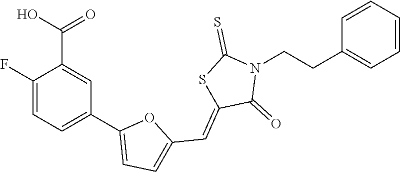 [SMILES: O=C(O)c1cc(-c2ccc(/C=C3\SC(=S)N(CCc4ccccc4)C3=O)o2)ccc1F] |
| 4 | 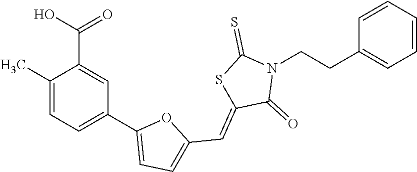 [SMILES: Cc1ccc(-c2ccc(/C=C3\SC(=S)N(CCc4ccccc4)C3=O)o2)cc1C(=O)O] |
| 5 | 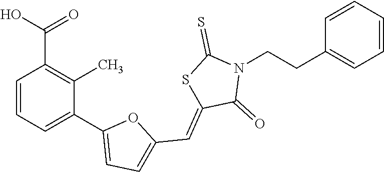 [SMILES: Cc1c(C(=O)O)cccc1-c1ccc(/C=C2\SC(=S)N(CCc3ccccc3)C2=O)o1] |
| 6 | 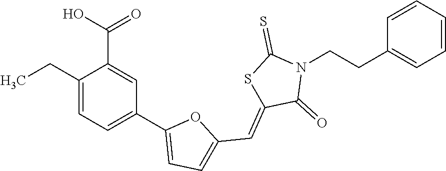 [SMILES: CCc1ccc(-c2ccc(/C=C3\SC(=S)N(CCc4ccccc4)C3=O)o2)cc1C(=O)O] |
| 7 | 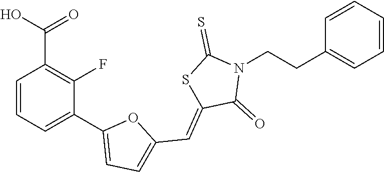 [SMILES: O=C(O)c1cccc(-c2ccc(/C=C3\SC(=S)N(CCc4ccccc4)C3=O)o2)c1F] |
| 8 | 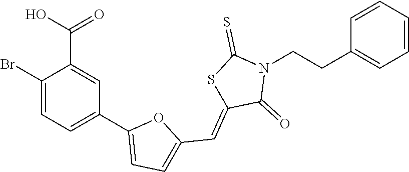 [SMILES: O=C(O)c1cc(-c2ccc(/C=C3\SC(=S)N(CCc4ccccc4)C3=O)o2)ccc1Br] |
| 9 | 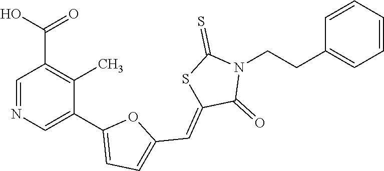 [SMILES: Cc1c(C(=O)O)cncc1-c1ccc(/C=C2\SC(=S)N(CCc3ccccc3)C2=O)o1] |
| 10 | 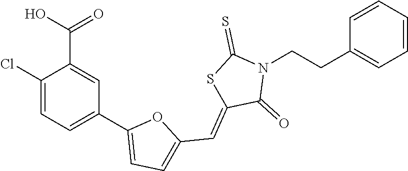 [SMILES: O=C(O)c1cc(-c2ccc(/C=C3\SC(=S)N(CCc4ccccc4)C3=O)o2)ccc1Cl] |
| 11 | 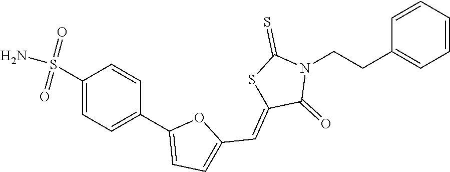 [SMILES: NS(=O)(=O)c1ccc(-c2ccc(/C=C3\SC(=S)N(CCc4ccccc4)C3=O)o2)cc1] |
| 12 | 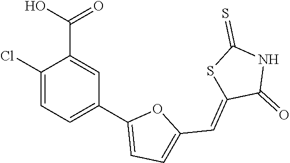 [SMILES: O=C1NC(=S)S/C1=C\c1ccc(-c2ccc(Cl)c(C(=O)O)c2)o1] |
| 13 | 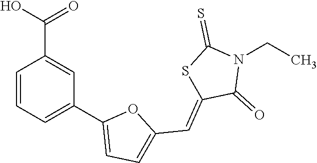 [SMILES: CCN1C(=O)/C(=C/c2ccc(-c3cccc(C(=O)O)c3)o2)SC1=S] |
| 14 | 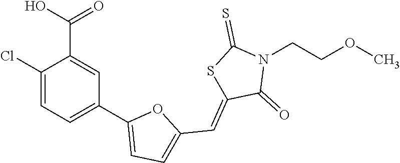 [SMILES: COCCN1C(=O)/C(=C/c2ccc(-c3ccc(Cl)c(C(=O)O)c3)o2)SC1=S] |
| 15 | 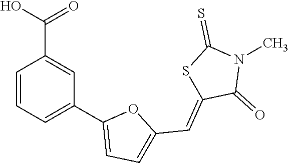 [SMILES: CN1C(=O)/C(=C/c2ccc(-c3cccc(C(=O)O)c3)o2)SC1=S] |
| 16 | 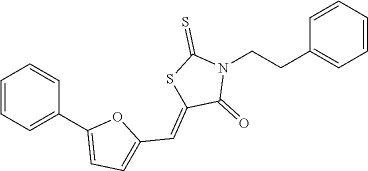 [SMILES: O=C1/C(=C/c2ccc(-c3ccccc3)o2)SC(=S)N1CCc1ccccc1] |
| 17 | 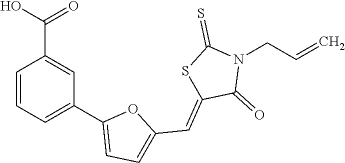 [SMILES: C=CCN1C(=O)/C(=C/c2ccc(-c3cccc(C(=O)O)c3)o2)SC1=S] |
| 18 | 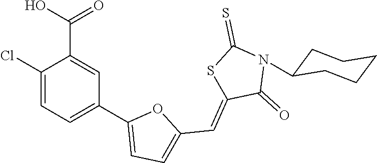 [SMILES: O=C(O)c1cc(-c2ccc(/C=C3\SC(=S)N(C4CCCCC4)C3=O)o2)ccc1Cl] |
| 19 | 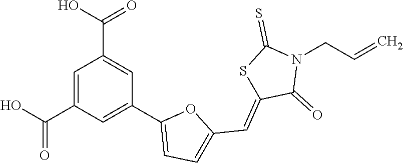 [SMILES: C=CCN1C(=O)/C(=C/c2ccc(-c3cc(C(=O)O)cc(C(=O)O)c3)o2)SC1=S] |
| 20 | 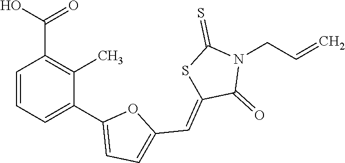 [SMILES: C=CCN1C(=O)/C(=C/c2ccc(-c3cccc(C(=O)O)c3C)o2)SC1=S] |
| 21 | 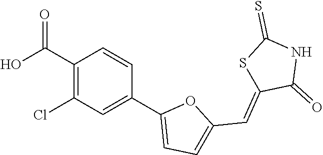 [SMILES: O=C1NC(=S)S/C1=C\c1ccc(-c2ccc(C(=O)O)c(Cl)c2)o1] |
| 22 | 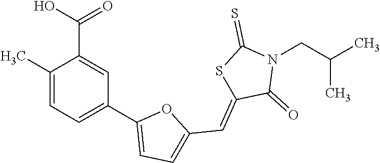 [SMILES: Cc1ccc(-c2ccc(/C=C3\SC(=S)N(CC(C)C)C3=O)o2)cc1C(=O)O] |
| 23 | 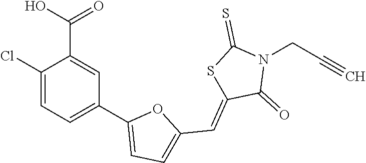 [SMILES: C#CCN1C(=O)/C(=C/c2ccc(-c3ccc(Cl)c(C(=O)O)c3)o2)SC1=S] |
| 24 | 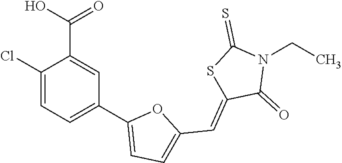 [SMILES: CCN1C(=O)/C(=C/c2ccc(-c3ccc(Cl)c(C(=O)O)c3)o2)SC1=S] |
| 25 | 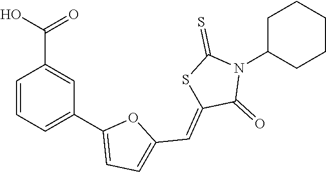 [SMILES: O=C(O)c1cccc(-c2ccc(/C=C3\SC(=S)N(C4CCCCC4)C3=O)o2)c1] |
| 26 | 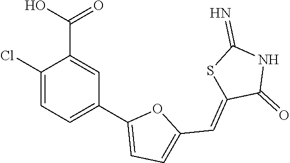 [SMILES: N=C1NC(=O)/C(=C/c2ccc(-c3ccc(Cl)c(C(=O)O)c3)o2)S1] |
| 27 | 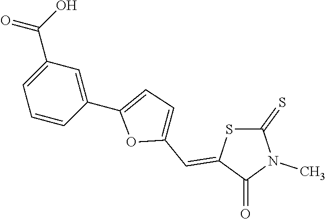 [SMILES: CN1C(=O)/C(=C/c2ccc(-c3cccc(C(=O)O)c3)o2)SC1=S] |
| 28 | 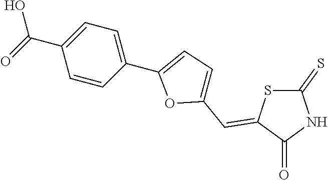 [SMILES: O=C1NC(=S)S/C1=C\c1ccc(-c2ccc(C(=O)O)cc2)o1] |

In some embodiments, disclosed herein are methods of treating or preventing a respiratory virus infection comprising administering to a person exposed to, or suspected of being exposed to, a respiratory virus a compound having a formula provided herein.

In some embodiments of the methods provided herein, the subject in need thereof has the respiratory infection confirmed by detection of a respiratory virus in a biological sample from the subject. In some embodiments, the respiratory virus is coronavirus. In some embodiments, the compound is a pancoronavirus inhibitor. In some embodiments, the coronavirus is MERS-CoV. In some embodiments, the coronavirus is SARS-CoV. In some embodiments, the coronavirus is SARS-CoV-2. In some embodiments, a compound disclosed herein inhibits all of the SARS-CoV-2 Alpha, Beta, and Delta variants.

In some embodiments, infection in a subject is diagnosed on the basis of exhibited symptoms. In other embodiments, infection is confirmed by detection of virus, or antibodies thereto, in a biological sample from the subject. In some embodiments, the biological sample is a blood sample (including a plasma or serum sample). In some embodiments, the biological sample is an oral or nasal swab or rinse. In some embodiments, the confirmatory test is a nucleic acid amplification test, such as a polymerase chain reaction-based test. In some embodiments, the confirmatory test is an antigen-based test, for example, an immunoassay.

In some embodiments of the above aspects, the emergence of symptoms in prevented or ameliorated. In other embodiments, the spread of infection beyond the respiratory tract is prevented or reduced. In further embodiments, infection or symptoms thereof are reduced or resolved more quickly than expected in an untreated infection. In some embodiments, administration of compound disclosed herein commences before the emergence of symptoms.

Disclosed herein are methods of treating respiratory virus infections. Non-limiting examples of respiratory viruses include influenza viruses, parainfluenza viruses, respiratory syncytial virus, coronaviruses, respiratory adenoviruses, enteroviruses, and metapneumovirus. Coronaviruses are a large family of ribonucleic acid (RNA) viruses which are enveloped with positive-sense single-stranded RNA genome and a nucleocapsid of helical symmetry. Coronaviruses have club-shaped spikes that project from their surface and which are the target of many vaccines and therapeutics. Exemplary coronaviruses include, but are not limited to rhinoviruses, severe acute respiratory syndrome-related coronaviruses (SARS-CoV and SARS-CoV-2) and Middle East respiratory syndrome coronavirus (MERS-CoV), and variants and mutants thereof. As used herein, the term “pancoronavirus” or “pancoronavirus inhibitor” refers to a compound that is active against at least SARS-CoV, SARS-CoV-2, and MERS-CoV.

In some embodiments of the above aspects, the coronavirus infection or exposure is by or to SARS-CoV infection. In other embodiments, the coronavirus infection or exposure is by or to SARS-CoV-2. In further embodiments, the coronavirus infection or exposure is by or to MERS-Cov.

In some embodiments related to infection by, or exposure to SARS-CoV-2, the SARS-CoV-2 is a variant, for example, B.1.1.7 UK (Alpha), B.1.351 RSA (Beta), B.1.617.2 India (Delta), P.1 Brazil (Gamma), B.1.427 and B.1.429 (California, USA), B.1.526 (New York, USA), Omicron (B.1.1.529), and the like. However, anti-viral activity of any compound disclosed herein is not limited to a known variant of a coronavirus. Compounds disclosed herein as being effective against a coronavirus are considered active against current variants and variants that may arise in the future absent statements to the contrary.

“Pharmaceutically acceptable salts” refers to the relatively non-toxic, inorganic and organic salts of compounds of the present disclosure. The compounds of the present disclosure are capable of reaction, for example, with a number of inorganic and organic bases to form pharmaceutically acceptable acid addition salts. Such pharmaceutically acceptable salts and common methodology for preparing them is described in P. Stahl, et al., Handbook of Pharmaceutical Salts: Properties, Selection and Use, (VCHA/Wiley-VCH, 2002); S. M. Berge, et al., “Pharmaceutical Salts,” Journal of Pharmaceutical Sciences, Vol 66, No. 1, January 1977, which is incorporated by reference herein for all it contains regarding pharmaceutically acceptable salts.

In some embodiments, the compounds or pharmaceutically acceptable salts thereof described herein are administered as a pharmaceutical composition in the methods provided herein.

In some embodiments, the compound is selected from Compounds 1-11 or 16, or a pharmaceutically acceptable salt thereof.

In some other embodiments, the compound is selected from Compounds 18 or 25, or a pharmaceutically acceptable salt thereof.

A pharmaceutical composition is one intended and suitable for the treatment of disease in humans. That is, it provides overall beneficial effect and does not contain amounts of ingredients or contaminants that cause toxic or other undesirable effects unrelated to the provision of the beneficial effect. A pharmaceutical composition will contain one or more active agents and may further contain solvents, buffers, diluents, carriers, and other excipients to aid the administration, solubility, absorption or bioavailability, and or stability, and the like of the active agent(s) or overall composition. A “pharmaceutically acceptable carrier, diluent, or excipient” is a medium generally accepted in the art for the delivery of biologically active agents to mammals, e.g., humans. The compounds of the present disclosure can be formulated as pharmaceutical compositions using a pharmaceutically acceptable carrier, diluent, or excipient and administered by a variety of routes. In particular embodiments, such compositions are for oral or intravenous administration. Such pharmaceutical compositions and processes for preparing them are well known in the art. See, e.g., Remington: The Science and Practice of Pharmacy (A. Gennaro, et al., eds., 19th ed., Mack Publishing Co., 1995), which is incorporated by reference for all it contains regarding pharmaceutical composition formulations.

The terms “treatment” “treating”, and the like, refer to the medical management of a patient with the intent to cure, ameliorate, or stabilize, a disease, pathological condition, or disorder. This term includes active treatment, that is, treatment directed specifically toward the improvement of a disease, pathological condition, or disorder, and may also include causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; and supportive treatment, that is, treatment employed to supplement another specific therapy directed toward the improvement of the associated disease, pathological condition, or disorder. Various embodiments may specifically include or exclude one or more of the above modes of treatment.

Aspects of the present specification provide, in part, administering a therapeutically effective amount of a compound or a composition disclosed herein. As used herein, the term “therapeutically effective amount” is synonymous with “therapeutically effective dose” and means at least the minimum dose of a compound or composition disclosed herein necessary to achieve a desired therapeutic effect. In some embodiments, it refers to an amount sufficient to prevent infection or the emergence of clinical symptoms. In other embodiments, it refers to an amount sufficient to halt or reverse the progression of infection or symptoms thereof. In still other embodiments, it refers to an amount sufficient to ameliorate symptoms. In further embodiments, an effective amount is one that speeds the clearance of infection or symptoms thereof. An effective dosage or amount of a compound or a composition disclosed herein can readily be determined by the person of ordinary skill in the art considering all criteria (for example, the rate of excretion of the compound or composition used, the pharmacodynamics of the compound or composition used, the nature of the other compounds to be included in the composition, the particular route of administration, the particular characteristics, history and risk factors of the individual, such as, e.g., age, weight, general health and the like, the response of the individual to the treatment, or any combination thereof) and utilizing his best judgment on the individual's behalf. Exemplary dosages are also disclosed herein above.

The term “treating” or “treatment” broadly includes any kind of treatment activity, including the diagnosis and mitigation of disease, or aspect thereof, in man or other animals, or any activity that otherwise affects the structure or any function of the body of man or other animals. Treatment activity includes the administration of the medicaments, dosage forms, and pharmaceutical compositions described herein to a patient, especially according to the various methods of treatment disclosed herein, whether by a healthcare professional, the patient his/herself, or any other person. Treatment activities include the orders, instructions, and advice of healthcare professionals such as physicians, physician's assistants, nurse practitioners, and the like, that are then acted upon by any other person including other healthcare professionals or the patient him/herself. Treatment activities include, for example, direction to the patient to undergo, or to a clinical laboratory to perform, a diagnostic procedure, such as a test to detect a coronavirus infection, or the particular variant or strain of the infecting virus, so that ultimately the patient may receive the benefit thereof including appropriate treatment.

A compound or a composition disclosed herein, can be administered using a variety of routes. Routes of administration suitable for treating a respiratory infection include, but are not limited to, oral administration, intravenous administration, subcutaneous administration, intramuscular administration, nasal administration, and inhalation to the lungs.

Oral compositions may include an inert diluent or an edible carrier. The oral compositions may be enclosed in gelatin capsules or compressed into tablets. Tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or sterites; a glidant such as colloidal silicon dioxide; a sweetening agent; and/or a flavoring agent.

Formulations suitable for intrapulmonary or nasal administration have a particle size for example in the range of about 0.1 to about 500 microns, such as about 0.5, about 1, about 30, or about 35 microns etc., which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents.

In certain aspects, parenteral, intradermal or subcutaneous formulations may be sterile injectable aqueous or oleaginous suspensions. Acceptable vehicles, solutions, suspensions and solvents may include, but are not limited to, water or other sterile diluent; saline; Ringer's solution; sodium chloride; fixed oils such as mono- or diglycerides; fatty acids such as oleic acid; polyethylene glycols; glycerine; propylene glycol or other synthetic solvents; antibacterial agents such as benzyl alcohol; antioxidants such as ascorbic acid; chelating agents such as ethylenediaminetetraacetic acid; buffers such as acetates, citrates or phosphates; and agents for the adjustment of tonicity such as sodium chloride or dextrose.

Solutions or suspensions used for parenteral, intradermal, or subcutaneous application may include one or more of the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation may be enclosed in ampoules, disposable syringes or multiple dose vials made of glass or plastic.

Pharmaceutical compositions suitable for injectable use may include sterile aqueous solutions or dispersions and sterile powders for the extemporaneous preparation of sterile injectable solutions or dispersion. For intravenous administration, suitable carriers include, but are not limited to, saline, bacteriostatic water, CREMOPHOR EL® (BASF, Parsippany, N.J.) or phosphate buffered saline (PBS). The solvent or dispersion medium may contain, for example, water, ethanol, polyol (for example, glycerol, propylene glycol, and liquid polyetheylene glycol, and the like), and suitable mixtures thereof. Proper fluidity can be maintained, for example, by the use of a coating such as lecithin, by the maintenance of the requited particle size in the case of dispersion and by the use of surfactants. Preventing growth of microorganisms can be achieved by various antibacterial and antifungal agents, for example, parabens, chlorobutanol, phenol, ascorbic acid, thimerosal, and the like. Composition of the present disclosure may also include isotonic agents such as, for example, sugars; polyalcohols such as manitol; sorbitol; or sodium chloride. Prolonged absorption of injectable compositions can be enhanced by addition of an agent which delays absorption, such as, for example, aluminum monostearate or gelatin.

In addition to oral or injected administration, systemic administration may be by transmucosal or transdermal means. For transmucosal or transdermal administration, penetrants may be used. Such penetrants are generally known in the art, and include, for example, detergents, bile salts, and fusidic acid derivatives. Transdermal administration may include a bioactive agent and may be formulated into ointments, salves, gels, or creams as generally known in the art. Transmucosal administration may be accomplished through the use of nasal sprays or suppositories.

The pharmaceutical compositions comprising the compounds disclosed herein may be administered in a therapeutically effective amount, according to an appropriate dosing regiment. As understood by a skilled artisan, an exact amount required may vary from subject to subject, depending on a subject's species, age and general condition, the severity of the infection, the particular agent(s) and the mode of administration. In some embodiments, about 0.001 mg/kg to about 50 mg/kg, of the pharmaceutical composition based on the subject's body weight is administered, one or more times a day, to obtain the desired therapeutic effect. In other embodiments, about 0.01 mg/kg to about 25 mg/kg, of the pharmaceutical composition based on the subject's body weight is administered, one or more times a day, to obtain the desired therapeutic effect.

A total daily dosage of the compounds and pharmaceutical compositions can be determined by the attending physician within the scope of sound medical judgment. A specific therapeutically effective dose level for any particular patient or subject will depend upon a variety of factors including the disorder being treated and the severity of the disorder; the activity of the specific compound employed; the specific composition employed; the age, body weight, general health, sex and diet of the patient or subject; the time of administration, route of administration, and rate of excretion of the specific compound employed; the duration of the treatment; drugs used in combination or coincidental with the specific compound employed, and other factors well known in the medical arts.

### EXAMPLES

### Example 1. Purification and Characterization of (Z)-2-Chloro-5-(5-((4-oxo-3-phenethyl-2-thioxothiazolidin-5-ylidene)methyl)furan-2-yl)benzoic Acid

Reagents and solvents were purchased from commercial suppliers and used without further purification unless otherwise stated. (Z)-2-chloro-5-(5-((4-oxo-3-phenethyl-2-thioxothiazolidin-5-ylidene)methyl)furan-2-yl)benzoic acid compounds were purchased from Sigma-Aldrich. Tetrahydrofuran (THF) was distilled from sodium-benzophenone under an argon atmosphere. Reaction progress was monitored using analytical thin-layer chromatography (TLC) on pre-coated silica gel GF254 plates (Macherey Nagel GmbH & Co. KG), and spots were detected under UV light (254 and 366 nm). Compounds were purified with flash column chromatography with a silica gel and particle size of 40-63 μM (Merck) as the stationary phase and hexane/ethyl acetate or dichloromethane/methanol mixtures as eluent systems. Nuclear magnetic resonance spectra were measured on a Bruker AV-400 NMR instrument (Bruker) in deuterated solvents (DMSO-d₆, CDCl₃, MeOD-d₄). Chemical shifts are expressed in ppm relative to DMSO-d₆ or MeOD-d₄ (2.50/3.31 for ¹H NMR; 39.52/49.00 for ¹³C NMR). The following abbreviations are used to set multiplicities: s=singlet, d=doublet, t=triplet, q=quartet, m =multiplet, br.=broad. Measurements for verification and purity of the compounds were performed by LC/MS. LC-MS/MS data were obtained using a Dionex Ultimate 3000 liquid chromatograph (Dionex) connected to an AB Sciex Qtrap 3200 mass spectrometer (AB Sciex,). LC separation was carried out on a Shim-pack GIST C18-AQ (150 mm×2.1 mm, 3 μm, Shimadzu) column. Mobile phase consisted of the mixture of 0.1% (v/v) formic acid in water (A) and acetonitrile (B). Separation was performed in isocratic mode 10%:90% (A:B). The mobile phase flow rate was 0.3 mL min⁻¹. The injection volume was 10 μL. Compounds were detected at λ=254 nm. All high resolution mass spectra (HRMS) were measured on AB Sciex TripleTOF 5600+ instrument equipped with DuoSpray (ESI) ion source. Samples were directly injected in the ion source in acetonitrile or methanol solutions acidified by formic acid. The melting points were measured in open capillaries and presented without correction.

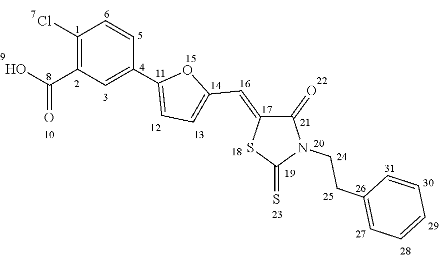 [SMILES: O=C(O)c1cc(-c2ccc(/C=C3\SC(=S)N(CCc4ccccc4)C3=O)o2)ccc1Cl]

(Z)-2-Chloro-5-(5-((4-oxo-3-phenethyl-2-thioxothiazolidin-5-ylidene)methyl)furan-2-yl)benzoic acid (Compound 1).

rt=2.087 min. Purity=98%. Mp=348-350° C. LC-MS: m/z [M+Na]+=492 Da.

¹H NMR (400.13 MHz, DMSO-d₆): δ=7.79 (d, J=2.4 Hz, 1 H, 6), 7.64 (s, 1 H, 3), 7.62 (dd, J=8.4 Hz, 2.2 Hz, 1 H, 5), 7.42-7.47 (m, J=3.5 Hz, 1 H, 16), 7.37 (d, J=3.5 Hz, 1 H, 13), 7.34 (d, J=3.5 Hz, 1 H, 12), 7.25-7.35 (m, 2 H, 27, 31), 7.18-7.24 (m, 3 H, 28, 29, 30), 4.23-4.32 (m, 2 H, 24), 2.90-3.02 (m, 2 H, 25).

¹³C NMR (100.61 MHz, DMSO-d₆): δ=193.88 (s, 1 C, 19), 168.94 (s, 1 C, 8), 166.56 (s, 1 C, 21), 157.82 (s, 1 C, 11), 149.46 (s, 1 C, 14), 144.65 (s, 1 C, 2), 137.87 (s, 1 C, 1), 130.41 (s, 1 C, 3), 130.13 (s, 1 C, 26), 128.93 (s, 2 C, 28, 30), 128.77 (s, 2 C, 27, 31), 126.85 (s, 1 C, 29), 124.31 (s, 1 C, 6), 123.66 (s, 1 C, 5), 123.46 (s, 1 C, 17), 123.27 (s, 1 C, 4), 118.44 (s, 1 C, 13), 118.43 (s, 1 C, 12), 110.71 (s, 1 C, 16), 45.50 (s, 1 C, 24), 32.37 (s, 1 C, 25).

HRMS (ESI): for C₂₃H₁₆CINO₄S_{2 [}M+Na]⁺: calcd 492.0102, found 492.0106

Purity: 100%. MW: 425.9593 (Calc.); 425.9 (found).

### Example 2. Fusion Inhibitors that Target Spike Protein of SARS-CoV-2 and Show Highly Potent Inhibition (Pancoronavirus) Against Related Coronaviruses

The spike protein of coronaviruses plays a critical role in virus binding to a cellular receptor, subsequent fusion to host cells, and entry to release its genetic material for continuing its life-cycle. The S2 subunit of the spike protein of coronaviruses is a Class I membrane fusion protein that belongs to many other enveloped viruses, such as HIV-1, influenza, Ebola, and the like, which all use a similar mechanism of membrane fusion. The fusion protein (FP) inserts in the host cell membrane and triggers the formation of a coiled-coil trimer by the heptad repeat region 1 (HR1). The HR2 binds to the trimer's hydrophobic groove in an antiparallel manner, creating a 6-HB, similar to what is seen with HIV-1 gp41-mediated fusion. However, there is no sequence homology between the HR1 and HR2 of coronaviruses spike protein with HR1 and HR2, respectively, of HIV-1 gp41. The formation of the 6-HB facilitates the virus membrane and host cell membrane to come closer for the fusion process to complete.

The reported crystallized post-fusion hairpin structure of SARS-CoV-2 (6LXT) and the similar SARS-CoV structure (1WYY) not only showed structural similarity but also indicated the presence of critical salt bridges between the HR1 and HR2 regions of both coronaviruses. Most noteworthy is that in SARS-CoV-2, K947 in the HR1 forms a salt bridge with E1182 of the HR2 domain. Similarly, in SARS-CoV, K929 forms a salt bridge with E1163. Earlier, a similar salt bridge interaction in the HIV-1 gp41 hairpin structure was reported, where K547 of the N-terminal heptad repeat region (NHR also known as HR1) region interacts with D632 of the C-terminal heptad repeat region (CHR also known as HR2) (He et al. J Virol, 82(22):11129-39, 2008). Furthermore, the design of a series of 3-(5-((4-oxo-3-phenethyl-2-thioxothiazolidin-5-ylidene)methyl)furan-2-yl)benzoic acid-based highly potent inhibitors of HIV-1 gp41 fusion, which contain a -COOH group, was also reported (Katritzky et al. J Med Chem 52(23):7631-7639, 2009).

Also, a computer-based docking of the compounds of the present disclosure (Compounds 1-28, Table 1) shows that the compounds interrupt the hairpin structure formation by interacting the -COOH group comprised in the compounds with the K547 residue and snuggly fitting to the hydrophobic groove created by three NHR regions (Katritzky et al., J Med Chem 52(23):7631-7639, 2009). Such similarities in the mechanism of virus fusion and involvement of salt bridges in forming the 6-HB in HIV-1 gp41 and coronaviruses suggest that this class of compounds may also interrupt the salt bridge formation by fitting in a cavity in the pre-fusion trimer structures of coronaviruses, prevent the 6-HB formation and eventually prevent fusion of viruses to host cells. Therefore, compounds of the present disclosure (Compounds 1-28, Table 1), were screened in a SARS-CoV-2, SARS-CoV, and MERS-CoV spike pseudotyped antiviral assay.

Experimental Methods

Cells and plasmids. The MRC-5, A549, HT-1080, Hela, HEK293T and HEK293T/17 cells were purchased from ATCC (Manassas, VA). The Human Lung carcinoma (A549) cells expressing Human Angiotensin-Converting Enzyme 2 (HA FLAG) (Catalog No. NR-53522) were obtained from BEI Resources, NIAID, NIH. The Human T-Cell Lymphoma Jurkat (E6-1) cells were obtained through the NIH ARP. The HuH-7 (JCRB0403) cells were obtained from JCRB Cell Bank (Osaka, Japan). The HT1080/ACE2 (human fibrosarcoma) cells, the 293T/ACE2 cells and the two plasmids pNL4-3ΔEnv-NanoLuc and pSARS-CoV-2-S_{Δ19} were kindly provided by Dr. P. Bieniasz of Rockefeller University (Schmidt et al. J Exp Med, 217(11), 2020). The pSV-A-MLV-Env (envelope) expression vector (Chang et al. Gene Ther 6(5):715-28, 1999; Landau et al. J Virol, 65(1):162-9, 1991) and the Env-deleted proviral backbone plasmids pNL4-3.Luc.R-E-DNA (Connor et al. Virol 206(2):935-944, 1995; He et al. J. Virol. 69(11):6705-6711, 1995) were obtained through the NIH ARP. The plasmids pSARS-CoV and pMERS-Cov were kindly provided by Dr. L. Du of New York Blood Center. The expression vectors containing SARS-CoV-2 full Spike wild-type (WT) gene from Wuhan-Hu-1 isolate (pUNO1-SARS-S) was purchased from InvivoGen. The pFB-Luc plasmid vector was purchased from Agilent Technologies.

Small Molecules. 3-(5-((4-oxo-3-phenethyl-2-thioxothiazolidin-5-ylidene)methyl) furan-2-yl)benzoic acids were used herein. The details of the synthesis, purification, and analytical characterization have been disclosed in Katritzky et al., J Med Chem 52(23):7631-7639, 2009. A control analog without the —COOH group was used as a comparative example, 5-((5-(4-chlorophenyl)furan-2-yl)methylene)-3-phenethyl-2-thioxothiazolidin-4-one (Compound 10) (purchased from Chembridge Corporation). Details of the analysis are described in Example 1. All purchased compounds had a purity >95%.

Pseudovirus preparation. To prepare pseudoviruses capable of single-cycle infection, 8×10⁶ HEK293T/17 cells were transfected with a proviral backbone plasmid and an envelope expression vector by using FuGENE HD (Promega) and following the manufacturer's instructions. To obtain the SARS-CoV-2, SARS-CoV and the MERS-CoV pseudoviruses, the cells were transfected with the HIV-1 Env-deleted proviral backbone plasmid pNL4-3ΔEnv-NanoLuc DNA and the pSARS-CoV-2-S_{Δ19}, pSARS-CoV and pMERS-CoV Env plasmids, respectively. For the A-MLV pseudovirus the cells were transfected with the Env-deleted proviral backbone plasmids pNL4-3.Luc.R-.E-DNA and the pSV-A-MLV-Env expression vector. Pseudovirus-containing supernatants were collected two days after transfection, filtered, tittered, and stored in aliquots at −80° C. Pseudovirus titers were determined to identify the 50% tissue culture infectious dose (TCID₅₀) by infecting the different cell types. For the titers in HT1080/ACE2 cells, 2×10⁴ cells were added to 100-μL aliquots of serial 2-fold dilutions of pseudovirus in a 96-well plate and incubated for 24 hr. For the titers in A549/ACE2 cells, 1×10⁴ cells were added to 100-μL aliquots of serial 2-fold dilutions of pseudovirus in a 96-well plate and incubated for 48 hr. For the titers in 293T/ACE2, MRC-5 and HUH-7 cells, 1×10⁴ cells/well were plated in a 96-well plate and incubated overnight before adding the 100-μL aliquots of serial 2-fold dilutions of pseudovirus and incubated for 48 h. Following the incubation time, the cells were washed with PBS and lysed with 50 μL of the cell culture lysis reagent (Promega). For the SARS-CoV-2 titers, 25 μL of the lysates were transferred to a white plate and mixed with the same volume of Nano-Glo® Luciferase reagent (Promega). For the A-MLV titers, 25 μL of the lysates were transferred to a white plate and mixed with 50μL of luciferase assay reagent (Luciferase assay system, Promega). The luciferase activity was immediately measured with a Tecan SPARK multifunctional microplate reader (Tecan). The wells producing relative luminescence unit (RLU) levels 10 times the cell background, were scored as positive. The TCID50 was calculated according to the Spearman-Karber method.

Analysis of the incorporation of the spike proteins into SARS-CoV-2, SARS-CoV and MERS-CoV pseudoviruses. To confirm the incorporation of the respective spike proteins into the SARS-CoV-2, SARS-CoV, and MERS-CoV pseudoviruses, 2 mL of the pseudovirus-containing supernatants were ultra-centrifuged for 2 hr at 40,000 rpm on a 20% sucrose cushion, to concentrate the viral particles. Viral pellets were lysed and processed for protein analysis. The viral proteins were resolved on a NuPAGE Novex 4-12% Bis-Tris Gel (Invitrogen). The SARS-CoV-2 and SARS-CoV viral lysates were immuno-detected with a SARS spike protein antibody (NB-100-56578, Novus Biological), followed by an anti-rabbit-IgG HRP linked whole antibody (GE Healthcare). The MERS-CoV viral lysate was immuno-detected with a MERS-coronavirus spike protein S2 polyclonal antibody (Invitrogen) followed by a donkey anti-rabbit IgG (H+L), HRP secondary antibody. Proteins were visualized using chemiluminescence.

Evaluation of the ACE2 and CD26 (DPP4) expression. The expression of the ACE2 receptor and the DDP4 receptor in the different cell lines was evaluated by Western Blot to find a correlation with the infection levels detected in the different cell lines (HT-1080\ACE2 and HT-1080, A549\ACE2, A549, 293T/ACE2, HEK293T and Hela for SARS-CoV-2 and SARS-CoV and HuH-7, MRC-5 and HeLa for MERS-CoV). Cell pellets were lysed and processed for protein analysis. For Blot-1, 50 μg of proteins (Media, Hela, HT1080 and HT1080/ACE2) was loaded, and for Blot-2, 75 μg of proteins (Media, Hela, A549 and A549/ACE2) was loaded. For Blot-3, 50 μg of proteins (MRC-5, HuH-7, HeLa and Media) was loaded. The proteins were resolved on a NuPAGE Novex 4-12% Bis-Tris Gel. Blot-1 and Blot-2 were immuno-detected with a human anti-ACE2 mAb (AC384) (Adipogen Life Sciences). The ECL Mouse IgG, HRP-linked whole Ab (from sheep) (Amersham) was used as a secondary antibody. Blot-3 was immuno-detected with a human DPP4 Monoclonal Antibody (OTI11D7), TrueMAB™ (Invitrogen) followed by a donkey anti-rabbit IgG (H+L), HRP secondary antibody. Cell lysates were also immuno-detected with the housekeeping gene β-actin as a loading control. Proteins were visualized using chemiluminescence.

Additionally, the correlation of SARS-CoV-2 and SARS-CoV pseudovirus infection with the expression of the ACE2 receptor was analyzed by infecting cells expressing different amounts of the ACE2 receptor, with the same volume of the pseudovirus-containing supernatant. Briefly, 50 μL of SARS-CoV-2 and SARS-CoV diluted with 50 μL serum free medium was added to wells of a 96-well cell culture plate. Next, the cells were added as follow: HT-1080\ACE2 and HT-1080 cells were added to the respective wells at the concentration of 2×10⁴ cells/well and incubated for 24 hr at 37° C.; A549\ACE2, A549 and Hela cells were added to the respective wells at a concentration of 1×10⁴ cells/well and incubated for 48 hr at 37° C. For the 293T/ACE2 and 293T, 1×10⁴ cell/well were plated the day before, then infected with the same volume of SARS-CoV-2 and SARS-CoV. Uninfected cells for all cell lines were used as negative control.

The correlation of infection of the MERS-CoV pseudovirus with the expression of the CD26 (DPP4) receptor was analyzed by infecting three different cell types (HuH-7, MRC-5, and Hela cells) with the same volume of the MERS-CoV pseudovirus-containing supernatant. Uninfected cells for all cell lines were used as negative control. Following the incubation time, the cells were washed with PBS and lysed with 50 μL of the cell culture lysis reagent. Twenty-five microliters of the lysates were transferred to a white plate and mixed with the same volume of Nano-Glo® Luciferase reagent. The luciferase activity was immediately measured with a Tecan SPARK.

Measurement of antiviral activity. The antiviral activity of the compounds of the present disclosure was evaluated in single-cycle infection assay by infecting different cell types with the SARS-CoV-2, SARS-CoV, or MERS-CoV pseudoviruses as previously described with minor modifications (Curreli et al. mBio 11(6), 2020); Nie et al. Emerg Microbes Infect 9(1):680-686, 2020).

HT1080/ACE2 cells. Briefly, in 96-well culture plates, aliquots of SARS-CoV-2 or SARS-CoV at about 3000 TCID₅ /well at a multiplicity of infection (MOI) of 0.1, were pre-incubated with escalating concentrations of the compounds of the present disclosure for 30 min. Next, 2×10⁴ cells were added to each well and incubated at 37° C. HT1080/ACE2 cells cultured with medium with or without the SARS-CoV-2 or SARS-CoV pseudoviruses were included as positive and negative controls, respectively. Following 24 hr incubation, the cells were washed with 200 μL of PBS and lysed with 50 μL of lysis buffer. Twenty-five microliters of the lysates were transferred to a white plate and mixed with the same volume of Nano-Glo® Luciferase reagent. The luciferase activity was measured immediately with the Tecan SPARK. The percent inhibition by the small molecules and the IC₅₀ (the half-maximal inhibitory concentration) values were calculated using the GraphPad Prism 9.0 software.

A549/ACE2 cells. For the evaluation of the antiviral activity in A549/ACE2 cells, aliquots of the pseudovirus SARS-CoV-2 or SARS-CoV at about 1500 TCID₅₀/well at a MOI of 0.1, were pre-incubated with escalating concentrations of the compounds of the present disclosure for 30 min. Next, 1×10⁴ cells were added to each well and incubated. A549/ACE2 cells cultured with medium with or without the SARS-CoV-2 or SARS-CoV pseudoviruses were included as positive and negative controls, respectively. Following 48 hr incubation, the cells were washed with PBS and lysed with 50 μL of lysis buffer. Twenty-five microliters of the cell lysates were processed as reported above to measure the luciferase activity and calculate the percent inhibition by the compounds of the present disclosure and the IC₅₀.

293T/ACE2 cells. The antiviral activity of the compounds of the present disclosure was evaluated in 293T/ACE2 cells infected with pseudoviruses SARS-CoV-2 and SARS-CoV. Briefly, 96-well plates were coated with 50 μL of poly-1-Lysine (Sigma-Aldrich, St. Louis, Mo.) at 50 μg/mL. Following 3 hr incubation at 37° C., the plates were washed with PBS and let to dry. The 293T/ACE2 cells were then plated at 1×10⁴/well and incubated overnight. On the following day, the aliquots of the pseudovirus at about 1500 TCID₅₀/well at a MOI of 0.1, were pretreated with graded concentrations of the compounds of the present disclosure for 30 min and added to the cells. 293T/ACE2 cells cultured with medium with or without the SARS-CoV-2 or SARS-CoV pseudoviruses were included as positive and negative controls, respectively. After 48 hr incubation, the cells were washed with PBS and lysed with 50 μL of lysis buffer. Twenty-five microliters of the cell lysates were processed as reported above to measure the luciferase activity and calculate the percent inhibition by the compounds of the present disclosure and the IC₅₀. Additionally, to test the specificity of the small molecules, their activity was evaluated against pseudovirus A-MLV at about 1500 TCID₅₀/well at a MOI of 0.1, by following the infection protocol described above. Following 48 hr incubation, 25 μL of the lysates were transferred to a white plate and mixed with 50 μL of luciferase assay reagent. The luciferase activity was immediately measured.

MRC-5 and HUH-7 cells. For the neutralization assay in MRC-5 and HUH-7 cells, 1×10⁴ cells/well were plated in 96-well cell culture plate and incubated overnight. On the following day, aliquots of the MERS-CoV pseudovirus at about 1500 TCID₅₀/well at a MOI of 0.1, were pretreated with graded concentrations of the small molecules for 30 min and added to the cells. MRC-5 and HUH-7 cells cultured with medium with or without the SARS-CoV-2 or SARS-CoV pseudoviruses were included as positive and negative controls, respectively. After 48 hr incubation, the cells were washed and lysed with 50 μL of lysis buffer (Promega). Twenty-five microliters of the cell lysates were processed as reported above to measure the luciferase activity and calculate the percent inhibition by the compounds of the present disclosure and the IC₅₀.

SARS-CoV-2 Microneutralization Assay. The standard live virus-based microneutralization (MN) assay was used. Briefly, serially two-fold and duplicate dilutions of individual compounds of the present disclosure were incubated with 120 plaque-forming units (PFU) of SARS-CoV-2 (US_WA-1/2020) at room temperature for 1 hr before transferring into designated wells of confluent Vero E6 cells (ATCC, CRL-1586) grown in 96-well cell culture plates. Vero E6 cells cultured with medium with or without the same amount of virus were included as positive and negative controls, respectively. After incubation at 37° C. for 3 days, individual wells were observed under the microscope to determine virus-induced formation of cytopathic effect (CPE). The efficacy of individual drugs was expressed as the lowest concentration capable of completely preventing virus-induced CPE in 100% of the wells.

Evaluation of cytotoxicity. The evaluation of the cytotoxicity of the compounds of the present disclosure in the different cell types was performed in parallel with the antiviral activity assay and measured by using the colorimetric CellTiter 96® AQueous One Solution Cell Proliferation Assay (MTS) (Promega) following the manufacturer's instructions.

HT1080/ACE2 cells. Briefly, aliquots of 100 μL of the compounds of the present disclosure at graded concentrations were incubated with 2×10⁴/well HT1080/ACE2 cells and cultured at 37° C. Following 24 hr incubation, the MTS reagent was added to the cells and incubated for 4 hr at 37° C. The absorbance was recorded at 490 nm. The percent of cytotoxicity and the CC₅₀ (the concentration for 50% cytotoxicity) values were calculated as above.

A549/ACE2 cells. For the cytotoxicity assay in A549/ACE2 cells, aliquots of escalating concentrations of the small molecules were incubated with 1×10⁴/well A549/ACE2 cells and cultured at 37° C. Following 48 hr incubation, the MTS reagent was added to the cells and incubated for 4 hr at 37° C. The absorbance was recorded and the percent of cytotoxicity and the CC₅₀ values were calculated as above.

HUH-7, MRC-5 and 293T/ACE2 cells. For the cytotoxicity assay in HUH-7, MRC-5 and 293T/ACE2 cells, 1×10⁴/well cells were plated in 96-well cell culture plate and incubated overnight. The following day, aliquots of escalating concentrations of the compounds of the present disclosure were added to the cells and incubated at 37° C. Following 48 hr incubation, the MTS reagent was added to the cells and incubated for 4 hr at 37° C. The absorbance was recorded at 490 nm. The percent of cytotoxicity and the CC₅₀ values were calculated as above.

Drug sensitivity of spike-mutated pseudovirus. Amino acid substitutions or deletions were introduced into the pSARS-CoV-2-S_{trunc} expression vector by site-directed mutagenesis (Stratagene) using mutagenic oligonucleotides as follows:

SaCoV2-E484K-F
(SEQ ID NO: 1)
ACCCCTTGTAACGGCGTGAAAGGCTTCAACTGCTACTTCCCA
SaCoV2-E484K-REV
(SEQ ID NO: 2)
TGGGAAGTAGCAGTTGAAGCCTTTCACGCCGTTACAAGGGG
SaCoV2-N501Y-F
(SEQ ID NO: 3)
TCCTACGGCTTTCAGCCCACATATGGCGTGGGCTATCAGCCC
SaCoV2-N501Y-REV
(SEQ ID NO: 4)
GGGCTGATAGCCCACGCCATATGTGGGCTGAAAGCCGTAGGA
SaCoV2-D614G-F
(SEQ ID N0: 5)
CAGGTGGCAGTGCTGTACCAGGGCGTGAACTGTACCGAAGTG
SaCoV2-D614G-REV
(SEQ ID NO: 6)
CACTTCGGTACAGTTCACGCCCTGGTACAGCACTGCCACCTG
SaCoV2-P681H-F
(SEQ ID NO: 7)
CAGACACAGACAAACAGCCACAGACGGGCCAGATCTGTG
SaCoV2-P681H-REV
(SEQ ID NO: 8)
CACAGATCTGGCCCGTCTGTGGCTGTTTGTCTGTGTCTG
SaCoV2-Δ(69-70)-S
(SEQ ID NO: 9)
GTGACCTGGTTCCACGCCATCTCCGGCACCAATGGCACCAAG
SaCoV2-Δ(69-70)-REV
(SEQ ID NO: 10)
CTTGGTGCCATTGGTGCCGGAGATGGCGTGGAACCAGGTCAC

Site mutations were verified by sequencing the entire spike gene of each construct. To obtain the SARS-CoV-2 pseudovirus carrying the amino acid substitutions, the cells were transfected with the HIV-1 Env-deleted proviral backbone plasmid pNL4-3ΔEnv-NanoLuc DNA and the mutant pSARS-CoV-2-S_{Δ19}as described above. Pseudoviruses were tittered by infecting 293T/ACE2 cells as described above. To measure the activity of the compounds against the pseudoviruses expressing different point mutations, 293T/ACE2 cells were infected with the ENV-mutated pseudoviruses pretreated for 30 min with different concentrations of the compounds of the present disclosure and incubated for 2 days, as described above. Cells were washed with PBS and lysed with 50 μL of cell culture lysis reagent. Twenty-five microliters of the cell lysates were processed as reported above to measure the luciferase activity and calculate the percent inhibition and the IC₅₀.

Cell-to-Cell fusion inhibition assay. For the SARS-CoV-2 mediated cell-to-cell fusion assay, Jurkat cells were used, which transiently expressed the luciferase gene and stably expressed the SARS-CoV-2 full Spike wild-type (WT) gene from Wuhan-Hu-1 isolate as donor cells and the 293T/ACE2 as acceptor cells. Briefly, Jurkat cells at 2×10⁵/mL were transfected with 1 μg/mL of SARS-CoV-2 WT expression vector, by using 5 μL/mL of FuGene HD and following the manufacturer's instructions. Following 24 hr incubation, transfected Jurkat cells were washed and selected for the SARS-CoV-2 spike expression using blasticidin at a concentration of 10 μg/m L. To rule out cell-resistance to the antibiotic, Jurkat cells that were not transfected with the SARS-CoV-2 spike, were exposed to the same concentration of blasticidin in parallel, the culture was completely depleted in about 14 days. The antibiotic was replaced every four days and the selection lasted for about 20 days. On the day before the assay, the 293T/ACE2 were plated in a 96-well cell culture plate at 8×10⁴/well, while the Jurkat cells were washed with PBS to remove the blasticidin, resuspended at 2×10⁵/mL and transfected with 1 μg/mL of pFB-Luc expression plasmid DNA using 5 μL/mL of FuGene HD. Following 20 hr incubation, the Jurkat cells were washed with PBS and aliquots of 8×10⁴/well were incubated with escalating concentrations of the compounds of the present disclosure for 1 hr. Finally, the Jurkat cells were transferred on the respective wells containing the 293T/ACE2 cells. Additionally, 293T/ACE2 cells cultured with medium with or without the Jurkart cells were included as positive and negative controls, respectively. As additional control, a set of 293T/ACE2 cells were incubated with Jurkat cells expressing the luciferase gene only (Jurkat-Luc). The plate was spun for 5 min at 1500 rpm and incubated for 4 hr at 37° C. To remove the Jurkat cells that did not fuse with the 293T/ACE2 cells, the wells were carefully washed twice with 200 μL of PBS. The cells were lysed to immediately measure the luciferase activity to calculate the percentage of inhibition of the SARS-CoV-2 mediated cell-to-cell fusion.

Binding analysis by SPR. The binding study of two of the most active small-molecules was performed by Profacgen. The bare gold-coated (thickness 47 nm) PlexArray Nanocapture Sensor Chip (Plexera Bioscience) was prewashed with 10× PBST for 10 min, 1× PBST for 10 min, and deionized water twice for 10 min before being dried under a stream of nitrogen prior to use. Various concentrations of biotinylated proteins dissolved in water were manually printed onto the chip with Biodo bioprinting at 40% humidity via biotin-avidin conjugation. Each concentration was printed in replicate, and each spot contained 0.2 μL of sample solution. The chip was incubated in 80% humidity at 4° C. overnight and rinsed with 10× PBST for 10 min, 1× PBST for 10 min, and deionized water twice for 10 min. The chip was then blocked with 5% (w/v) non-fat milk in water overnight, and washed with 10× PBST for 10 min, 1× PBST for 10 min, and deionized water twice for 10 min before being dried under a stream of nitrogen prior to use. SPRi measurements were performed with PlexAray HT (Plexera Bioscience). Collimated light (660 nm) passes through the coupling prism, reflects off the SPR-active gold surface, and is received by the CCD camera. Buffers and samples were injected by a non-pulsatile piston pump into the 30 μL flowcell that was mounted on the coupling prism. Each measurement cycle contained four steps: washing with PBST running buffer at a constant rate of 2 μL/s to obtain a stable baseline, sample injection at 5 μL/s for binding, surface washing with PBST at 2 μL/s for 300 s, and regeneration with 0.5% (v/v) H₃PO₄ at 2 μL/s for 300 s. All the measurements were performed at 25° C. The signal changes after binding and washing (in AU) are recorded as the assay value.

Selected protein-grafted regions in the SPR images were analyzed, and the average reflectivity variations of the chosen areas were plotted as a function of time. Real-time binding signals were recorded and analyzed by Data Analysis Module (DAM, Plexera Bioscience). Kinetic analysis was performed using BlAevaluation 4.1 software (Biacore, Inc.).

In vivo pharmacokinetics in rats. Two of the most active compounds, Compound 1 and Compound 2, were selected to evaluate the pharmacokinetics (PK) in rats. Rats were 11 weeks and 1 day old and weighed between 200-250 grams. A total of twelve female Sprague Dawley (SD) rats (Charles River Laboratory) were implanted with a jugular vein catheter and were assigned to the study following acclimation for seven days. Rats were divided into four (4) treatment groups consisting of three rats each. On Day 0, 10 mg/kg/animal was administered via oral gavage for groups 1 and 3. On Day 0, 5 mg/kg/animal was administered via tail vein injection based on their body weights for groups 2 and 4. All animals underwent blood collection for plasma at 5 min, 15 min, 30 min, 1 hr, 2 hr, 4 hr, 8 hr, and 24 hr post-dosing. At 24-hr post-dosing, all animals were euthanized post terminal blood collection without performing a necropsy. The study was conducted under BSL-1 safety conditions.

The concentrations of the test article (compound) in plasma were determined using high-performance liquid chromatography with tandem mass spectrometric detection (LC-MS/MS). The test article was isolated by liquid-liquid extraction. A partial aliquot of the supernatant was transferred to a clean 96-well collection plate, evaporated to dryness under nitrogen, and reconstituted with water. The extracted samples were analyzed using a Sciex 5500 mass spectrometer. The quantitative range of the assay was from 1-2,000 ng/mL. Analysis of pharmacokinetic parameters—pharmacokinetic parameters were calculated using PkSolver. Graphs were generated using PkSolver.

Equilibrium solubility. The equilibrium solubility of one test article was measured in pH 7.4 aqueous buffer. The buffer was prepared by combining 50 mL of 0.2 M KH₂PO₄ with 150 mL of H₂O, and then adjusting to pH 7.4 with 10 N NaOH. At least 1 mg of powder for each test article was combined with 1 mL of buffer to make a 1 mg/mL mixture. The samples were shaken on a Thermomixer® overnight at room temperature. The samples were then passed through a 0.45 pm PTFE syringe filter. The filtrate was sampled and diluted in duplicate 10-, 100-, 1000-, and 10000-fold into a mixture of 1:1 buffer:acetonitrile (ACN) prior to analysis. All samples were assayed by LC-MS/MS using electrospray ionization against standards prepared in a mixture of 1:1 assay buffer:ACN. Standard concentrations ranged from 1.0 μM to 0.3 nM. Solubility results are presented in Table 2.

TABLE 2

| | Solubility in Phosphate Buffer (μM) | | |
|---|---|---|---|
| Test Article | R1 | R2 | Average |
| Compound 1 | 4.35 | 5.08 | 4.72 |

P-GP Substrate Assessment. Caco-2 cells (clone C2BBe1) were obtained from American Type Culture Collection. Cell monolayers were grown to confluence on collagen-coated, microporous membranes in 12-well assay plates. The permeability assay buffer was Hanks' balanced salt solution (HBSS) containing 10 mM HEPES and 15 mM glucose at a pH of 7.4. The buffer in the receiver chamber also contained 1% bovine serum albumin. The dosing solution concentration was 5 μM of test article in the assay buffer +/−1 μM valspodar. Cells were first pre-incubated for 30 min with HBSS containing +/−1 μM valspodar. Cell monolayers were dosed on the apical side (A-to-B) or basolateral side (B-to-A) and incubated at 37° C. with 5% CO₂ in a humidified incubator. Samples were taken from the donor and receiver chambers at 120 min. Each determination was performed in duplicate. The flux of lucifer yellow was also measured post-experimentally for each monolayer to ensure no damage was inflicted to the cell monolayers during the flux period. All samples were assayed by LC-MS/MS using electrospray ionization. The apparent permeability (Papp) and percent recovery were calculated as follows and presented in Tables 3 and 4:

Papp=(dCr/dt)×Vr/(A∴CA) (1)

Percent Recovery=100×(ₗVr×C^{final})+(Vd×Cd^{final}))/(Vd×CN) (2)

### Where,

dCr/dt is the slope of the cumulative receiver concentration versus time in μM s⁻¹;
Vr is the volume of the receiver compartment in cm³;
Vd is the volume of the donor compartment in cm³; A is the area of the insert (1.13 cm² for 12-well);
CA is the average of the nominal dosing concentration and the measured 120 min donor concentration in μM;
CN is the nominal concentration of the dosing solution in μM;
C^{final} is the cumulative receiver concentration in μM at the end of the incubation period;
Cd^{final} is the concentration of the donor in μM at the end of the incubation period.
Efflux ratio (ER) is defined as Papp (B-to-A)/Papp (A-to-B).

TABLE 3

| | | Acceptance Criteria |
|---|---|---|
| Plates | 12-well | |
| Passage Number | 67 | |
| Age at QC (days) | 20 | |
| Age at Experiment (days) | 27 | |
| Atenolol Papp, 10⁻⁶ cm/s | 0.193 | ≤0.5 |
| Propranolol Papp, 10⁻⁶ cm/s | 19.7 | 10-30 |
| Digoxin A-to-B Papp, 10⁻⁶ cm/s | 0.340 | N/A^{a} |
| Digoxin B-to-A Papp, 10⁻⁶ cm/s | 12.9 | N/A^{a} |
| Digoxin Efflux Ratio | 37.9 | ≥10 |

^{a}Not Available

TABLE 4

| | | Recovery | Papp (10⁻⁶ cm/s) | | | Efflux | P-gp Substrate |
|---|---|---|---|---|---|---|---|
| Test Article | Direction | (%) | R1 | R2 | AVG | Ratio | Classification |
| Compound 1 | A-to-B | 46.2 | 18.1 | 15.6 | 16.9 | 1.21 | Negative |
| | B-to-A | 54.2 | 17.8 | 23.1 | 20.4 | | |
| Compound 1 | A-to-B | 34.4 | 20.4 | 20.6 | 20.5 | 1.15 | |
| +1 μM Valspodar | B-to-A | 56.6 | 20.9 | 26.4 | 23.6 | | |

P-gp Substrate Classification:
CER ≥ 1.0 without valspodar, and reduced by ≥ 50% with valspodar: Positive
CER ≥ 1.0 without valspodar, and reduced by < 50% with valspodar: Negative
CER < 1.0 without and with valspodar: Negative
CER = Corrected Efflux Ratio = ER − 1

Plasma Protein Binding. Studies were carried out in mixed-gender human plasma, obtained from BiolVT and collected on K2EDTA. A Pierce Rapid Equilibrium Dialysis (RED) device was used for all experiments. Stock solutions of the test article and control compound were first prepared in DMSO. Aliquots of the DMSO solutions were dosed into 1.0 mL of plasma at a dosing concentration of 5 μM for the test article and 10 μM for the co-dosed control compound, warfarin. Plasma (300 μL), containing test article and control compound, was loaded into two wells of the 96-well dialysis plate. Blank phosphate-buffered saline (PBS) (500 μL) was added to each corresponding receiver chamber. The device was then placed into an enclosed heated rocker that was pre-warmed to 37° C., and allowed to incubate for four hours. After 4 hours of incubation, both sides were sampled.

Aliquots (50 μL for donor, 200 μL for receiver) were removed from the chambers and placed into a 96-well plate. Plasma (50 μL) was added to the wells containing the receiver samples, and 200 μL of PBS was added to the wells containing the donor samples. Two volumes of acetonitrile (ACN) were added to each well, and the plate was mixed and then centrifuged at 3,000 rpm for 10 min. Aliquots of the supernatant were removed, diluted 1:1 into water, and analyzed by LC-MS/MS.

Protein binding values were calculated as follows: % Bound=[(PARR in Donor—PARR in Receiver)/(PARR in Donor)]×100 PARR=peak area response ratio to internal standard, including applicable dilution factors and results presented in Table 5.

TABLE 5

| | % Bound | | | | | |
|---|---|---|---|---|---|---|
| | Test Article | | | Warfarin | | |
| Test Article | R1 | R2 | Average | R1 | R2 | Average |
| Compound 1 | >99.5 | >99.5 | >99.5 | 98.4 | 98.2 | 98.3 |

Warfarin binding acceptance criteria: ≥98.0% bound

Stability in Liver Microsomes. Mixed-gender human liver microsomes (Lot# 1010420) were purchased from XenoTech. The reaction mixture, minus NADPH, was prepared as described below. The test article was added into the reaction mixture at a final concentration of 1 μM. The control compound, testosterone, was run simultaneously with the test article in a separate reaction. The reaction mixture (without cofactor) was equilibrated in a shaking water bath at 37° C. for 5 min. The reaction was initiated by the addition of the cofactor, and the mixture was incubated in a shaking water bath at 37° C. Aliquots (100 μL) were withdrawn at 0, 15, 30, 60, 90, and 120 min. Test article and testosterone samples were immediately combined with 400 μL of ice-cold 50/50 acetonitrile (ACN)/H₂O containing 0.1% formic acid and internal standard to terminate the reaction. The samples were then mixed and centrifuged to precipitate proteins. All samples were assayed by LC-MS/MS using electrospray ionization. Analytical conditions are outlined in Appendix 1. The peak area response ratio (PARR) to internal standard was compared to the PARR at time 0 to determine the percent remaining at each time point. Half-lives and clearance were calculated using GraphPad software, fitting to a single-phase exponential decay equation and presented in Tables 6 and 7.

TABLE 6

| | | | | | | | | Clᵢₙₜ |
|---|---|---|---|---|---|---|---|---|
| | % Remaining of Initial (n = 1) | | | | | | Half- | (mL/min/ |
| | 0 | 15 | 30 | 60 | 90 | 120 | life | mg |
| Test Article | min | min | min | min | min | min | (min) | protein) |
| Compound 1 | 100 | 101 | 80.5 | 68.9 | 69.5 | 41.6 | 112 | 0.0124 |

TABLE 7

| | | | Clᵢₙₜ | Acceptable |
|---|---|---|---|---|
| Control | | Half-life | (ml/min/mg | Range |
| Compound | Species | (min) | protein) | (t_{1/2}, min) |
| Testosterone | Human | 11.3 | 0.122 | ≤41 |

CYP IC₅₀. The test articles, at eight concentrations (0-10 μM), were incubated with pooled HLM (0.25 mg protein/mL) in phosphate buffer (100 mM, pH 7.4) containing MgCl₂ (5 mM), NADPH (1 mM), and an individual CYP probe substrate (at approximately Km). The reaction mixture minus NADPH was equilibrated in a shaking water bath at 37° C. for 5 min. The reaction was initiated by the addition of NADPH, followed by incubation at 37° C. for 10-30 min depending on the individual CYP isoform. The reaction was terminated by the addition of two volumes of ice-cold acetonitrile. Negative (vehicle) controls were conducted without the test article. Positive controls were performed in parallel at a single concentration using known CYP inhibitors. After the removal of protein by centrifugation at 1640×g (3000 rpm) for 10 min at 4° C., the supernatants were transferred to a 96-well plate and diluted with water containing internal standard (stable isotope-labeled CYP probe metabolite). The formation of CYP probe metabolite was determined by LC-MS/MS and the results presented in Tables 8-10.

TABLE 8
CYP Probe Substrates and Metabolites

| | | | Incubation | |
|---|---|---|---|---|
| CYP | Probe Substrate | Metabolite | Time (min) | Positive Control Inhibitor |
| CYP1A2 | Phenacetin (63 μM) | Acetaminophen | 20 | α-Naphthoflavone (1 μM) |
| CYP2B6 | Bupropion (75 μM) | OH bupropion | 20 | Thio-TEPA (30 μM) |
| CYP2C8 | Amodiaquine (2 μM) | Desethylamodiaquine | 20 | Montelukast (5 μM) |
| CYP2C9 | Diclofenac (10 μM) | 4’-OH diclofenac | 20 | Sulfaphenazole (10 μM) |
| CYP2C19 | S-mephenytoin (40 μM) | 4’-OH mephenytoin | 30 | (+)-N-3-benzylnirvanol (5 μM) |
| CYP2D6 | Bufuralol (7 μM) | T-OH bufuralol | 20 | Quinidine (1 μM) |
| CYP3A | Midazolam (2.5 μM) | T-OH midazolam | 10 | Ketoconazole (1 μM) |
| | Testosterone (55 μM) | 6β-OH testosterone | 10 | Ketoconazole (1 μM) |

TABLE 9

| | % of Control Enzyme Activity (n = 1)^{a} | | | | | | | | |
|---|---|---|---|---|---|---|---|---|---|
| | 0 | 0.0137 | 0.0412 | 0.123 | 0.370 | 1.11 | 3.33 | 10 | |
| CYP | μM | μM | μM | μM | μM | μM | μM | μM | IC₅₀ (μM) |
| CYP1A2 | 100 | 99.2 | 96.7 | 93.8 | 92.2 | 89.8 | 69.5 | 41.3 | 7.40 |
| CYP2B6 | 100 | 97.5 | 98.1 | 90.4 | 94.2 | 76.9 | 47.0 | 15.2 | 3.19 |
| CYP2C8 | 100 | 102 | 89.0 | 98.3 | 87.8 | 76.9 | 25.3 | 3.87 | 2.08 |
| CYP2C9 | 100 | 99.4 | 98.2 | 97.2 | 95.1 | 85.1 | 59.9 | 29.8 | 5.01 |
| CYP2C19 | 100 | 103 | 110 | 101 | 102 | 92.0 | 70.8 | 40.7 | 7.31 |
| CYP2D6 | 100 | 95.8 | 98.7 | 98.6 | 101 | 97.0 | 84.3 | 59.2 | >10 |
| CYP3A | 100 | 96.7 | 97.0 | 94.5 | 95.1 | 95.9 | 88.2 | 61.6 | >10 |
| (Midazolam) | | | | | | | | | |
| CYP3A | 100 | 96.6 | 97.0 | 94.8 | 96.3 | 94.6 | 82.2 | 59.9 | >10 |
| (Testosterone) | | | | | | | | | |

^{a}Percent of control enzyme activity = 100 × (Enzyme activity in the presence of TA/Enzyme activity in the absence of TA). Enzyme activity was calculated from the peak area ratio of CYP probe metabolite to internal standard by LC-MS/MS.

TABLE 10

| Positive Control Inhibitor | CYP | IC₅₀ (μM) |
|---|---|---|
| α-Naphthoflavone | CYP1A2 | 0.0164 |
| Thio-TEPA | CYP2B6 | 4.22 |
| Montelukast | CYP2C8 | 0.750 |
| Sulfaphenazole | CYP2C9 | 0.595 |
| (+)-N-3-benzylnirvanol | CYP2C19 | 0.324 |
| Quinidine | CYP2D6 | 0.0641 |
| Ketoconazole | CYP3A (Midazolam) | 0.0450 |
| Ketoconazole | CYP3A (Testosterone) | 0.0270 |

### Results

Validation of the Pseudoviruses

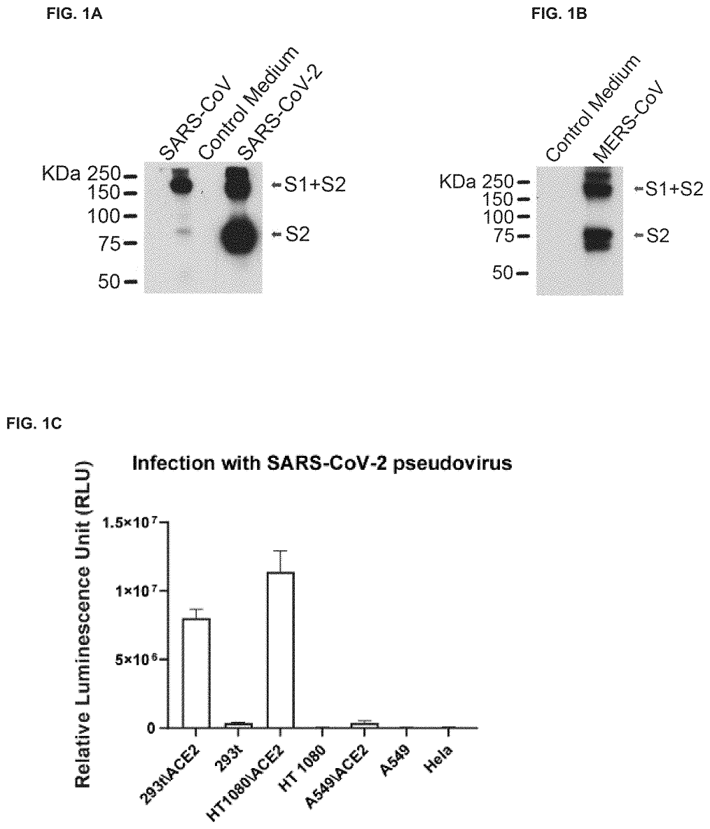

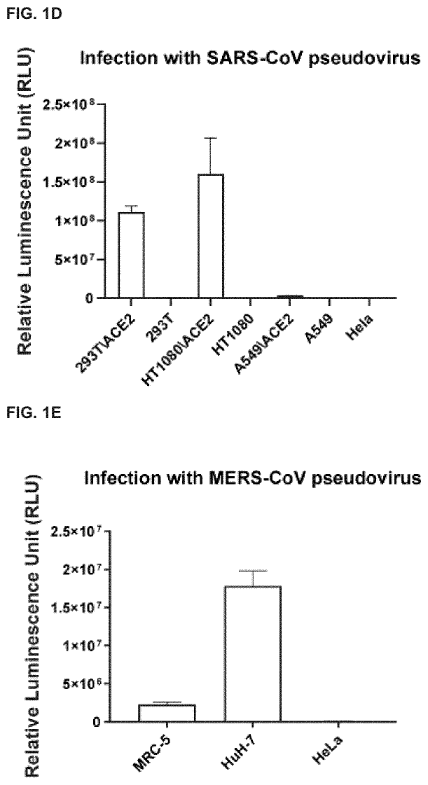

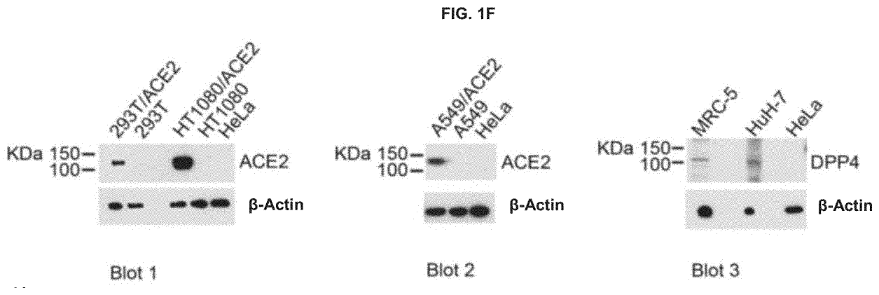

SARS-CoV-2, SARS-CoV, and MERS-CoV pseudoviruses were prepared, and found to be capable of single-cycle infection by transfecting HEK293T/17 cells with an HIV-1 Env-deleted proviral backbone plasmid pNL4-3ΔEnv-NanoLuc and the respective spike plasmid. The incorporation of the spike proteins in the respective pseudoviruses was then validated by Western blot analysis. A SARS spike protein antibody (Novus Biologicals) was used which targets the spike protein S2 of SARS-CoV-2, with SARS-CoV and SARS-CoV-2 pseudovirus lysates (FIG. 1A). Similarly, a MERS-coronavirus spike protein S2 polyclonal antibody (Invitrogen) was used, which targets the spike protein S2 of the MERS-CoV pseudovirus, to validate the incorporation of the MERS-CoV spike protein (FIG. 1B). For SARS-CoV-2, a specific band at 80 kDa was found, which identifies the subunit S2 and a second band at about 190 kDa, which corresponds to the full-length S protein (S1+S2). In SARS-CoV pseudovirus lysate, the same antibody immunodetected a lighter band at 80 kDa representing S2 subunit and a 190 kDa band, which corresponds to the full-length of the spike S protein (FIG. 1A). Additionally, for MERS-CoV lysates, the subunit S2 was identified at 75 kDa and the full-length S protein at about 185 kDa (FIG. 1B). These analyses confirmed the correct incorporation of the spike proteins in the respective pseudoviruses.

Afterward, the correlation of SARS-CoV-2 and SARS-CoV pseudovirus infection levels was analyzed with the expression levels of the hACE2 receptor by infecting three different cell types, which overexpress the ACE2 receptor. The human kidney 293T/ACE2 cells, the human fibrosarcoma HT1080/ACE2 cells, and the human lung carcinoma cells A549/ACE2 and the respective parental cell types HEK293T cells, HT1080 cells, and A549 cells were used. Also, HeLa cells were infected, which do not express the hACE2 receptor, as a control. The cells were exposed to the same volumes of the supernatant containing the respective pseudoviruses. Both pseudoviruses did not infect the HeLa cells (FIGS. 1C-1D). Further, a low level of infection of the parental cell lines HEK293T, HT1080, and A549 was detected with the SARS-CoV-2 pseudovirus compared to the respective related cell types overexpressing the ACE2 receptor. The 293T/ACE2 and HT1080/ACE2 cells supported high levels of SARS-CoV-2 infection. About 8×10⁶ RLU and 1.1×10⁷ RLU were detected, respectively, which correspond to a 24-fold and 490-fold higher infection than what was detected for the parental cell type HEK293T and HT1080 respectively. The infection detected for the A549/ACE2 cells was moderate (about 3.8×10⁵ RLU) compared to HT1080/ACE2 and 293T/ACE2 cells and about 13-fold higher than what was detected for the parental cell type A549. For the SARS-CoV infection study, similar results were obtained. These results were generally consistent with the expression levels of the ACE2 receptor in the different cell lines by western blot (FIG. 1F). As shown in Blot-1, ACE2 expression was undetectable in the parental cell lines 293T and HT1080 while it was overexpressed in HT1080/ACE2 cells. A lower amount of ACE2 was detected in the 293T/ACE2 cell lysate with respect to the amounts detected in HT1080/ACE2 cells consonant with the infection study findings (FIGS. 1C-1D). The lower infection detected in A549/ACE2 cells suggested a lower ACE2 expression in these cells. For this reason, in Blot-2 (FIG. 1F), to visualize the ACE2 expression, a higher concentration of proteins (75 μg) was loaded and a 2× concentration of antibodies was used. These data, taken together, confirmed that SARS-CoV-2 and SARS-CoV pseudoviruses infect the cells through their interaction with the ACE2 receptor.

To analyze the correlation of MERS-CoV pseudovirus infection levels with the DPP4 (CD26) receptor expression, the fibroblast cell line from lung MRC-5 and hepatocyte-derived carcinoma cell line HUH-7 was infected; as a control, HeLa cells which do not express the DPP4 receptor, were also infected. In this case, the cells were exposed to the same volumes of the supernatant containing the MERS-CoV pseudovirus. The HuH-7 cells were found to support an 8.6-fold higher level of MERS-CoV infection than the MRC-5 cells. About 2×10⁷ RLU and 2.3×10⁶ RLU were detected, respectively. No infection of the HeLa cells (FIG. 1E) was observed. The expression levels of the DPP4 receptor in the two cell lines are reported in Blot-3 (FIG. 1F). These data, taken together, confirmed that the spike proteins were correctly incorporated in the respective pseudoviruses and that these pseudoviruses infected the cells through their interaction with the respective receptors.

Antiviral Activity and Cytotoxicity in a Pseudovirus Assay

The anti-coronavirus activity of the small molecules (Compounds 1-28) was evaluated by infecting three cell types overexpressing the hACE2 receptor, the human kidney cells 293T/ACE2, the human fibrosarcoma cells HT1080/ACE2, and the human lung cells A549/ACE2, with aliquots of the SARS-CoV-2 pseudovirus which was pretreated with escalating concentrations of the compounds for 30 min. The concentration of the compounds required to inhibit 50% (IC₅₀) of SARS-CoV-2 pseudovirus infection was calculated, and the results are shown in Table 11.

Most of the compounds of the present disclosure were found to inhibit SARS-CoV-2 infection with nanomolar (nM) activity. The only exceptions were Compound 5 which had micromolar activity (1205±240 nM, 1050±252 nM, and >2000 nM in 293T/ACE2, HT1080/ACE2, and A549/ACE2 cells, respectively), and Compound 10 which showed no antiviral activity at 2000 nM. Compound 1, Compound 2, and Compound 4 were the most potent compounds and consistently inhibited the SARS-CoV-2 infection with low nanomolar activity in the three cell lines. For Compound 1, the calculated IC₅₀s were in the range of 32.3-63.4 nM, and the selectivity index (SI obtained from the ratio CC₅₀/IC₅₀) ranged from 755-2755; for Compound 2, the calculated IC₅₀s were in the range of 22.8-58 nM, and the SIs varied from 1630->4000; finally, for Compound 4 the calculated the IC₅₀s were in the range of 26-73 nM, and the obtained SIs ranged from >1370->2096. Compound 3 also displayed potent anti-SARS-CoV-2 activity, but the IC₅₀s and SIs obtained for the three cell lines were slightly higher than those detected for Compound 1, Compound 2, and Compound 4 (IC₅₀s: 60.1-120 and SIs:750->1563). Compounds 6-9 showed lower potency than Compound 1-4, as shown by the higher IC₅₀s and SIs. Remarkably, all the compounds displayed better activity when tested in 293T/ACE2 and HT1080/ACE2 cells than when tested in A549/ACE2 cells and consistently showed an increase of the IC₅₀s and SIs. The cytotoxicity (CC₅₀) of the small molecules was assessed in parallel with the inhibition assays (Table 11), and these values were used to determine the SIs. As noted, in some cases (HT1080/ACE2 and A549/ACE2 cell lines), the small molecules did not induce any apparent cell toxicity at the highest dose tested (Table 11). When the cells (rather than the virus) were pretreated with the compounds of the present disclosure for 30 min before infection, the cells did not confer any protection against SARS-CoV-2 infection even at the higher dose used in the assay (2000 nM). Without being bond by theory, it is believed that the target of these compounds is virus-related and not cell-related.

To derive a Structure-activity Relationship (SAR), it was observed that Compound 2, which does not have any para-substituent in the carboxyphenyl ring, showed the best antiviral potency against SARS-CoV2. When there is a hydrophobic substituent at the para position of that ring, Compound 1, Compound 4, Compound 8, and Compound 18 showed excellent antiviral activity. Compound 3 has a highly electronegative fluoro atom, and it showed somewhat reduced activity compared to Compound 2 and Compound 4. With a bulkier hydrophobic substituent (—CH₂CH₃), Compound 6 showed slightly lower antiviral activity. Furthermore, a substituent at the ortho position in the carboxy phenyl ring was not well tolerated and showed poor antiviral activity. The introduction of nitrogen in the phenyl ring (pyridine) did not improve the antiviral activity, although the solubility of this molecule may have improved. Since it was hypothesized that the —COOH group of these compounds might be interacting with one of the key positively charged residues in the HR1 region to interfere with the 6-HB formation, an analog devoid of the —COOH group (Compound 10) was further tested. Compound 10 showed no antiviral potency at the highest dose tested. The SAR was extended by substituting the phenylethyl group with either no substitution in the rhodanine moiety (Compound 12 and Compound 28) or substituting with a smaller hydrophobic group (prop-1-yne, Compound 23). Furthermore, the position of —COOH is also critical. When the -COOH group is at the para position of the phenyl ring (Compound 28), it loses the inhibitory activity. The data, although with few compounds, clearly generated an insightful SAR. Furthermore, both Compound 18 and Compound 25, which differ by the presence or absence of the -Cl group on the ortho position of the -COOH group of the phenyl moiety, displayed antiviral potency against SARS-CoV2, although Compound 18 had higher potency.

Although Compound 12 and Compound 23 contain -COOH and -Cl groups at the same positions as Compound 1, analysis showed that Compound 12 and Compound 23 did not display any activity at the highest dose tested. This data underscores the fact that a combination of electrostatic and appropriate hydrophobic interactions is essential for the antiviral potency of this series of compounds. The mere presence of the ene-rhodanine moiety shows no role in the inhibition process. Similarly, Compound 28 had a carboxylic group at the para position, had no hydrophobic group attached to the NH of the rhodanine moiety, and showed no inhibition. If the ene-rhodanine scaffold has any role in the antiviral potency through its promiscuous nature, it should have shown potent antiviral activity because the rhodanine scaffold has no steric hindrance to bind non-specifically to random protein targets.

| | 293T/ACE2 cells | | | HT1080/ACE2 cells | | | A549/ACE2 cells | | |
|---|---|---|---|---|---|---|---|---|---|
| Compound | IC₅₀ (nM)^{a} | CC₅₀ (μM)^{a} | SI | IC₅₀ (nM)^{a} | CC₅₀ (μM)^{a} | SI | IC₅₀ (nM)^{a} | CC₅₀ (μM)^{a} | SI |
| 1 | 51 ± 17 | 37.5 ± 1 | 755 | 32.3 ± 4.6 | 89 ± 2 | 2755 | 63.6 ± 4.6 | 86 ± 8.7 | 1352 |
| 2 | 22.8 ± 0.8 | 37.5 ± 2 | 1630 | 25.3 ± 0.6 | >100 | >4000 | 58 ± 1.7 | >100 | >1724 |
| 3 | 60.1 ± 8.5 | 45 ± 5 | 750 | 64 ± 18 | >100 | >1563 | 120 ± 5 | >100 | >833 |
| 4 | 26 ± 1 | 40.7 ± 2.3 | 1565 | 47.7 ± 16 | >100 | >2096 | 73 ± 4.1 | >100 | >1370 |
| 5 | 1205 ± 240 | 35 ± 2 | 29 | 1050 ± 252 | >100 | >95 | >2000 | >100 | N/A^{b} |
| 6 | 185 ± 5.8 | 40 ± 6 | 216 | 245 ± 5 | >100 | >408 | 613 ± 72 | >100 | >163 |
| 7 | 298 ± 60 | 45 ± 0.4 | 151 | 290 ± 57 | >100 | >345 | 416 ± 25 | >100 | >240 |
| 8 | 65.8 ± 6.2 | 38.7 ± 1.2 | 586 | 94 ± 9 | >100 | >1063 | 254 ± 27 | >100 | >394 |
| 9 | 342 ± 46 | 33.7 ± 2.5 | 98 | 367 ± 73 | >100 | >274 | 596 ± 42 | >100 | >168 |
| 10 | >2000 | 64 ± 14 | N/A^{b} | >2000 | >100 | N/A^{b} | N/A^{b} | N/A^{b} | N/A^{b} |
| 11 | 31 ± 0.2 | 13.3 ± 2 | 429 | 25 ± 0.2 | 10.5 ± 1 | N/A^{b} | N/A^{b} | N/A^{b} | N/A^{b} |
| 12 | >4000 | >100 | N/A^{b} | >4000 | >100 | N/A^{b} | N/A^{b} | N/A^{b} | N/A^{b} |
| 13 | 92 ± 24 | 74 ± 3 | 804 | 176 ± 1 | >100 | N/A^{b} | N/A^{b} | N/A^{b} | N/A^{b} |
| 14 | 867 ± 110 | 81 ± 1 | 97 | 860 ± 248 | >100 | N/A^{b} | N/A^{b} | N/A^{b} | N/A^{b} |
| 15 | 154 ± 8 | 53 ± 8 | 526 | 166 ± 44 | >100 | N/A^{b} | N/A^{b} | N/A^{b} | N/A^{b} |
| 16 | 231 ± 62 | 75 ± 7 | 229 | 592 ± 112 | 40 ± 2 | N/A^{b} | N/A^{b} | N/A^{b} | N/A^{b} |
| 17 | 322 ± 60 | >100 | 233 | 283 ± 96 | >100 | N/A^{b} | N/A^{b} | N/A^{b} | N/A^{b} |
| 18 | 13.4 ± 1.8 | >100 | >7463 | 16 ± 2.6 | >100 | N/A^{b} | N/A^{b} | N/A^{b} | N/A^{b} |
| 19 | >4000 | >100 | N/A^{b} | >4000 | >100 | N/A^{b} | N/A^{b} | N/A^{b} | N/A^{b} |
| 20 | 111.3 ± 3.5 | >100 | >898 | 199 ± 12 | >100 | N/A^{b} | N/A^{b} | N/A^{b} | N/A^{b} |
| 21 | >4000 | >100 | N/A^{b} | >4000 | >100 | N/A^{b} | N/A^{b} | N/A^{b} | N/A^{b} |
| 22 | >4000 | 68 ± 4 | N/A^{b} | >4000 | 70 ± 4 | N/A^{b} | N/A^{b} | N/A^{b} | N/A^{b} |
| 23 | >4000 | >100 | N/A^{b} | >4000 | 92 ± 3 | N/A^{b} | N/A^{b} | N/A^{b} | N/A^{b} |
| 24 | 111 ± 6.6 | 78 ± 3 | 703 | 175 ± 16 | >100 | N/A^{b} | N/A^{b} | N/A^{b} | N/A^{b} |
| 25 | 36.3 ± 6.7 | >100 | >2755 | 39 ± 13 | >100 | N/A^{b} | N/A^{b} | N/A^{b} | N/A^{b} |
| 26 | >4000 | >100 | N/A^{b} | N/A^{b} | N/A^{b} | N/A^{b} | N/A^{b} | N/A^{b} | N/A^{b} |
| 27 | 200 ± 11 | >100 | >500 | 148 ± 85 | >100 | >675 | N/A^{b} | N/A^{b} | N/A^{b} |
| 28 | >4000 | 83 ± 4 | N/A^{b} | >4000 | ~100 | N/A^{b} | N/A^{b} | N/A^{b} | N/A^{b} |

^{a}The reported IC₅₀ and CC₅₀ values represent the means ± standard deviations (n = 3).
^{b}Not Available

To assess whether the small molecules of the present disclosure (Compounds 1-28) have pancoronavirus antiviral activity, their inhibitory activity against the SARS-CoV pseudovirus was tested (Table 12). In this assay, the most potent compounds were found to be Compounds 1-4. Compounds 1-3 had excellent IC₅₀s of 13.8-17 nM (SI: 2265-2717) in 293T/ACE2 cells, 19.3-39 nM (SI: 2282->5181) in HT1080/ACE2 cells and 98-157 nM (SI: >637->901) in A549/ACE2 cells. Compound 4 had slightly higher IC₅₀s in 293T/ACE2 cells and HT1080/ACE2 cells (SI: 509 and >1852, respectively), but it displayed the second best activity and best SI (>1000) in A549/ACE2 cells with respect to Compounds 1-3. Compound 5 had poor activity against this pseudovirus in the three cell lines tested. Also, Compounds 6-9, displayed anti-SARS activity in all cell lines, but these compounds were less potent and displayed lower consistency than Compounds 1-4. In the case of SARS-CoV, the SAR followed a similar pattern to what was observed with SARS-CoV-2, thus due to the presence of the amino acid and high level of structural similarity of the S2 domain of the spike protein in these viruses.

| | 293T/ACE2 cells | | HT1080/ACE2 cells | | A549/ACE2 cells | |
|---|---|---|---|---|---|---|
| Compound | IC₅₀ (nM)^{a} | SI | IC₅₀ (nM)^{a} | SI | IC₅₀ (nM)^{a} | SI |
| 1 | 17 ± 2.6 | 2265 | 39 ± 8.2 | 2282 | 98 ± 4 | 878 |
| 2 | 13.8 ± 0.2 | 2717 | 19.3 ± 1.1 | >5181 | 111 ± 9 | >901 |
| 3 | 17.8 ± 4 | 2528 | 25.7 ± 0.6 | >3891 | 157 ± 12.5 | >637 |
| 4 | 80 ± 2 | 509 | 54 ± 3.5 | >1852 | 100 ± 13 | >1000 |
| 5 | 1853 ± 179 | 19 | 1760 ± 330 | >57 | >2000 | N/A^{b} |
| 6 | 175 ± 7 | 229 | 133 ± 9.5 | >752 | 867 ± 62 | >115 |
| 7 | 111 ± 1.7 | 405 | 52 ± 8.9 | >1923 | 271 ± 76 | >369 |
| 8 | 128 ± 6.7 | 302 | 225 ± 26 | >444 | 700 ± 70 | >143 |
| 9 | 217 ± 22 | 155 | 246 ± 10 | >407 | 350 ± 15 | >286 |

^{a}The reported IC₅₀ values represent the means ± standard deviations (n = 3).
^{b}Not Available

Compounds 1-9 were also evaluated for activity against MERS-CoV pseudovirus by infecting the HUH-7 cells (a differentiated hepatocyte-derived carcinoma cell line) and the MRC-5 cell (a fibroblast cell line from the lung) (Table 13A and 13B). As depicted in Table 13A, Compounds 1-4 were found to be the most potent compounds against MERS-CoV pseudovirus in both cell lines (IC₅₀: 95-158 nM in HuH-7 cells and IC₅₀ : 76.5-123 nM in MRC-5 cells and SIs>582 and >407, respectively). Compound 9 also had a significant anti-MERS-CoV inhibitory activity with ICₛₒ of about 200 nM in both cell lines. Compound 5 had no MERS-CoV inhibitory activity at the highest dose used in this assay (2000 nM) in both cell lines, while Compounds 6-8 inhibited MERS-CoV infection with an IC₅₀ lower than 1000 nM. Hence, most of the compounds of the present disclosure possess a pancoronavirus predisposition. Similar SAR with the MERS-CoV was observed.

| | HUH-7 cells | | | MRC-5 cells | | |
|---|---|---|---|---|---|---|
| Compound | IC₅₀ (nM)^{a} | CC₅₀ (μM)^{a} | SI | IC₅₀ (nM)^{a} | CC₅₀ (μM)^{a} | SI |
| 1 | 95 ± 22 | ~100 | 1053 | 76.5 ± 0.3 | 69 ± 1 | 902 |
| 2 | 112 ± 7 | 80 ± 1 | 714 | 77 ± 0.5 | 63 ± 2.8 | 818 |
| 3 | 158 ± 14 | 92 ± 2 | 582 | 123 ± 17 | >50 | >407 |
| 4 | 131 ± 6 | 80 ± 6 | 611 | 80 ± 18 | 63.5 ± 3.5 | 794 |
| 5 | >2000 | 76 ± 2 | N/A^{b} | >2000 | 72.5 ± 1 | N/A^{b} |
| 6 | 569 ± 31 | 71 ± 1 | 125 | 945 ± 77 | 71 ± 2.8 | 75 |
| 7 | 933 ± 153 | N/A^{b} | N/A^{b} | 927 ± 63.5 | 68.8 ± 1 | 74 |
| 8 | 509 ± 43 | 83 ± 2 | 163 | 559 ± 66 | 67.5 ± 3.5 | 121 |
| 9 | 214 ± 23 | 55 ± 9 | 257 | 232 ± 17 | 42.5 ± 3.5 | 183 |

^{a}The reported IC₅₀ and CC₅₀ values represent the means ± standard deviations (n = 3).
^{b}Not Available

| | HuH-7/MERS-CoV | | |
|---|---|---|---|
| Compound | IC₅₀ (nM)^{a} | CC₅₀ (μM)^{a} | SI |
| 11 | 13.2 ± 3.7 | 30 ± 3.5 | 2273 |
| 18 | 10.5 ± 0.9 | >100 | >9524 |
| 20 | 340 ± 112 | 84 ± 4 | 247 |
| 24 | 88 ± 13 | ~100 | 1136 |
| 25 | 247 ± 47 | >100 | 405 |
| 27 | 96 ± 14 | >100 | 1042 |

^{a}The reported IC₅₀ and CC₅₀ values represent the means ± standard deviations (n = 3)

To verify the specificity of the compounds of the present disclosure for coronaviruses, the compounds were evaluated against the amphotropic murine leukemia virus (A-MLV), which enters the cells via micropinocytosis. It was found that none of the compounds showed appreciable activity against this control pseudovirus (IC₅₀>783 nM) (Table 14). Based on these experiments, the data suggest that their inhibitory activity is specific to the coronaviruses. However, Compounds 1-9 also showed HIV-1 fusion inhibitory activity.

| | 293T-ACE2 cells/A-MLV |
|---|---|
| Compound | IC₅₀ (nM) |
| 1 | 1397 ± 12 |
| 2 | 783 ± 76 |
| 3 | 960 ± 57 |
| 4 | 1623 ± 115 |
| 5 | >2000 |
| 6 | >2000 |
| 7 | >2000 |
| 8 | >2000 |
| 9 | 1627 ± 118 |

^{a}The reported IC₅₀ values represent the means ± standard deviations (n = 3)

Inhibition of the Replication-Competent SARS-CoV-2 (US_WA-1/2020)

The antiviral activity of the compounds of the present disclosure was evaluated by exposing Vero E6 cells with the replication-competent authentic virus SARS-CoV-2 (US_WA-1/2020). On the third day post-infection, the cells were observed under the microscope to evaluate the formation of virus-induced cytopathic effect (CPE). The efficacy of the small molecules was expressed as the lowest concentration capable of completely prevent virus-induced CPE (IC100). Compound 1 and Compound 2 were found to be the most efficient compounds in preventing the complete formation of CPEs with an IC₁₀₀ of 1.25 μM followed by Compound 3, Compound 4, and Compound 9, which completely prevented the formation of the virus-induced CPEs at 2.5 μM. Compounds 7 and 8 also prevented the formation of CPEs with an IC₁₀₀ of 5 μM while Compounds 5 and 6 did not completely prevent the formation of the virus-induced CPE at 10 μM, which was the highest dose used in this assay (Table 15). These findings support the results obtained with the single-cycle pseudovirus-based antiviral assays.

| | Vero cells/ |
|---|---|
| | SARS-CoV-2 |
| Compound | IC₁₀₀ (μM)^{a} |
| 1 | 1.25 |
| 2 | 1.25 |
| 3 | 2.5 |
| 4 | 2.5 |
| 5 | >10 |
| 6 | >10 |
| 7 | 5 |
| 8 | 5 |
| 9 | 2.5 |

^{a}Values indicate the lowest concentration capable of completely preventing virus-induced CPE in 100% of the wells.

Neutralization of B.1.1.7 UK (Alpha), B.1.351 RSA (Beta), and B.1.617.2 India (Delta) SARS-CoV-2 Variants

Coronaviruses, like other RNA viruses, depend on an error-prone RNA-dependent RNA polymerase to facilitate virus replication and adaptation. Currently, the emergence of major SARS-CoV-2 variants carrying multiple mutations in their spike is causing new concerns for increased virulence and reduced vaccine efficacy. The potency of the compounds of the present disclosure was evaluated against the B.1.1.7 United Kingdom (UK), the B.1.351 South African (RSA) SARS-CoV-2, and B.1.617.2 India (Delta) variants carrying single or multiple key spike mutations (B.1.1.7 UK variant: 69-70 deletion (Δ69-70)/N501Y/P681H; B.1.351 RSA: E484K/N501Y/D614G; and B.1.617.2 Delta: D614G/P681R/D950N) (Table 16). Introduction of single-, double-, and triple-mutations was carried out into the pSARS-CoV-2-S_{trunc} expression vector. Next, 293T/ACE2 cells were infected with WT and mutant SARS-CoV-2 pseudoviruses in the absence or in the presence of Compounds 1-4. Compound 5 was used as a control because of its poor activity against the coronaviruses. Compound 1 was found to have potent antiviral activity against all mutant pseudoviruses carrying single-, double-, or triple-mutations of the three variants B.1.1.7 UK, B.1.351 RSA, and B.1.617.2 Delta, as indicated by the low IC₅₀s detected, which were similar to the IC₅₀ obtained for the SARS-CoV-2 WT pseudovirus (Table 16). Compound 2 was also a highly potent inhibitor against all mutant pseudoviruses even though the IC₅₀s detected for the SARS-CoV-2 WT pseudovirus was lower than those detected against the mutant pseudoviruses (IC₅₀ in the range 35-88.7 nM for all the pseudovirus variants). Compound 4 was highly potent against all the variants, exhibited a significant increase of the IC₅₀ was against the B.1.1.7 UK triple mutant variant Δ69-70/N501Y/P681H (IC₅₀ of 158 nM) and the B.1.617.2 Delta single, double and triple mutant variants (IC₅₀ of 148-239 nM). A significant increase of the IC₅₀ was obtained against the B.1.1.7 UK triple mutant variant 469-70/N501Y/P681H (IC₅₀ of 158 nM), but the compound retained appreciable activity against this mutant. Compound 3 was slightly less efficient against all the pseudoviruses tested, including the WT. In this case, a higher IC₅₀ was obtained when tested against the B.1.1.7 UK triple mutant variant: Δ69-70/N501Y/P681H (IC₅₀ of 232 nM). Finally, Compound 5 had poor/no activity against these variants. These results, taken together, indicate that the compounds maintain their potency against the three mutant SARS-CoV-2 variants.

| | Compound 1 | Compound 2 | Compound 3 | Compound 4 | Compound 5 |
|---|---|---|---|---|---|
| | IC₅₀ (nM)^{a} | IC₅₀ (nM)^{a} | IC₅₀ (nM)^{a} | IC₅₀ (nM)^{a} | IC₅₀ (nM)^{a} |
| SARS-CoV-2 | | | | | |
| WT | 51 ± 17 | 22.8 ± 0.8 | 60.1 ± 8.5 | 26 ± 1 | 1205 ± 240 |
| B.1.1.7 UK | | | | | |
| N501Y | 52.5 ± 0.2 | 49 ± 0.5 | 44.5 ± 0.5 | 51 ± 0.4 | >2000 |
| A69-70 | 48 ± 3 | 55 ± 0.5 | 69 ± 7 | 53 ± 0.5 | >2000 |
| P681H | 35 ± 0.5 | 51 ± 0.3 | 55 ± 0.3 | 44 ± 0.4 | 1720 ± 215 |
| N501Y/A69-70 | 46 ± 0.5 | 66 ± 1 | 94 ± 16 | 53 ± 11 | >2000 |
| N501Y/A69-70/P681H | 57 ± 0.5 | 79 ± 14 | 232 ± 17 | 158 ± 31 | >2000 |
| B.1.351 RSA | | | | | |
| E484K | 42 ± 0.7 | 45 ± 0.2 | 80 ± 10.6 | 45 ± 0.4 | >2000 |
| D614G | 56 ± 0.6 | 61 ± 0.3 | 145 ± 28 | 58 ± 0.4 | 1730 ± 62.5 |
| N501Y/D614G | 57 ± 2 | 59 ± 13 | 104 ± 1 | 77 ± 29 | >2000 |
| E484K/N501Y | 32 ± 0.5 | 36 ± 0.4 | 42 ± 1 | 51 ± 5 | >2000 |
| E484K/D614G | 33 ± 0.5 | 35 ± 0.2 | 44 ± 0.8 | 44 ± 3 | 1846 ± 124 |
| E484K/N501Y/D614G | 45 ± 2 | 43 ± 0.5 | 101 ± 2 | 43 ± 2.5 | >2000 |
| B.1.617.2 India (Delta) | | | | | |
| D950N | 47.7 ± 15 | 88.7 ± 6 | N/A^{b} | 207 ± 29 | >2000 |
| D614G/P681R | 49 ± 13 | 65 ± 18 | N/A^{b} | 148 ± 30 | >2000 |
| D614G/D950N | 33 ± 10 | 26 ± 2 | N/A^{b} | 77.5 ± 16.5 | >2000 |
| D614G/P681R/D95ON | 58 ± 7 | 53 ± 12 | N/A^{b} | 239 ± 7 | >2000 |

^{a}The reported IC₅₀ values represent the means ± standard deviations (n = 3).
^{b}Not Available.

Inhibition of SARS-CoV-2 Mediated Cell-to-Cell Fusion.

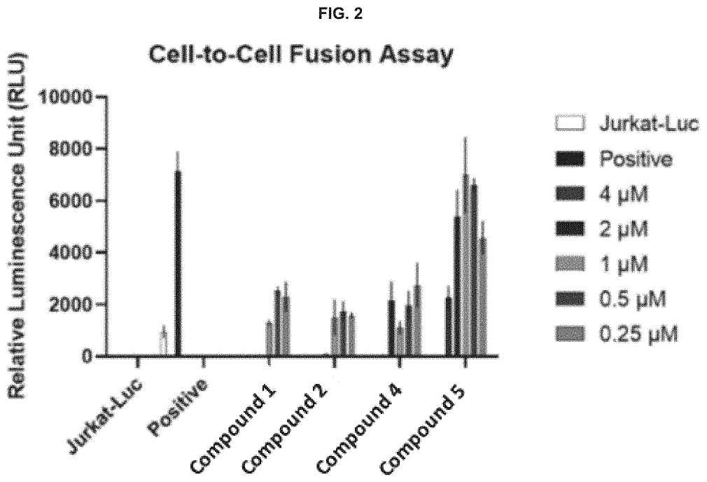

Efficient virus spreading can be achieved by either a cell-free or a cell-associated mode involving direct cell-to-cell contact/fusion. Cell-to-cell fusion mode permits the virus to infect adjacent cells without producing free virus, contributing to tissue damage and inducing the formation of syncytia. ACE2/SARS-CoV-2 spike interaction and subsequent conformational changes in the spike protein are critical in initiating the fusion of membranes of infected cells with the adjacent cells. Since it was observed that the compounds of the present disclosure inhibit SARS-CoV-2 in single cycle assay and bind to the SARS-CoV-2 S trimer, the most potent compounds, Compounds 1, 2, and 4 were investigated to determine whether or not these compounds could prevent SARS-CoV-2 mediated cell-to-cell fusion. Compound 5 was used as a negative control, as it was showed that it had poor anti-SARS-CoV-2 activity. A new and novel cell-to-cell fusion assay was set up which uses Jurkat cells expressing the luciferase gene and the SARS-CoV-2 spike wild type-WT as donor cells and 293T/ACE2 as acceptor cells. Jurkat cells was have chosen because the cells grow in suspension, and if not fused with the 293T/ACE2 cells, Jurkat cells can easily be removed from the wells by washing twice with PBS. Jurkat cells were pretreated with escalating concentrations of the compounds for 1 hr, then added to the 293T/ACE2 cells and co-cultured for 4 hr to allow fusion. It was observed that Compound 5 only inhibited the SARS-CoV-2 mediated cell-to-cell fusion at the higher dose (4 μM) (FIG. 2), while Compound 1, Compound 2, and Compound 4 potently inhibited the cell-to-cell fusion even at the lowest dose used in this assay. In fact, at the concentration of 250 nM, Compound 1, Compound 2, and Compound 4 still maintained a 62-79% inhibitory activity of the SARS-CoV-2 indicating that these small molecules interfere with the SARS-CoV-2 S-mediated cell-to-cell fusion.

Binding Affinity of the Two Most Potent Inhibitors by SPR Analysis

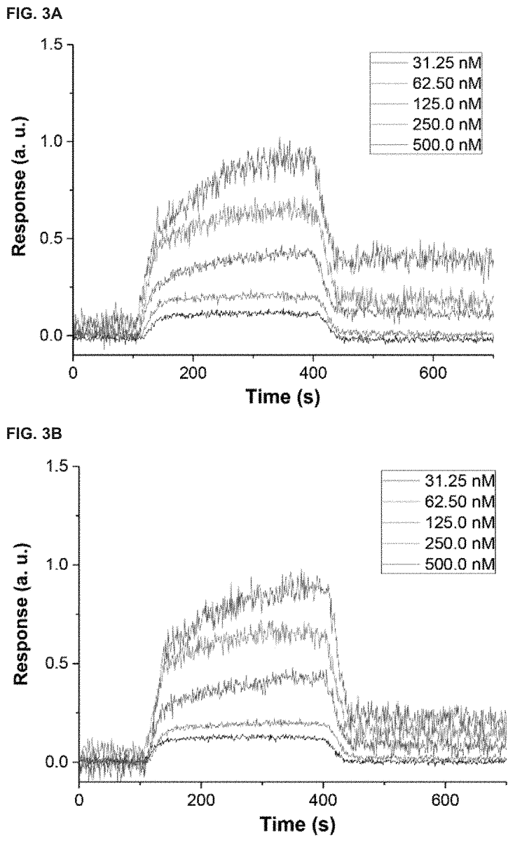

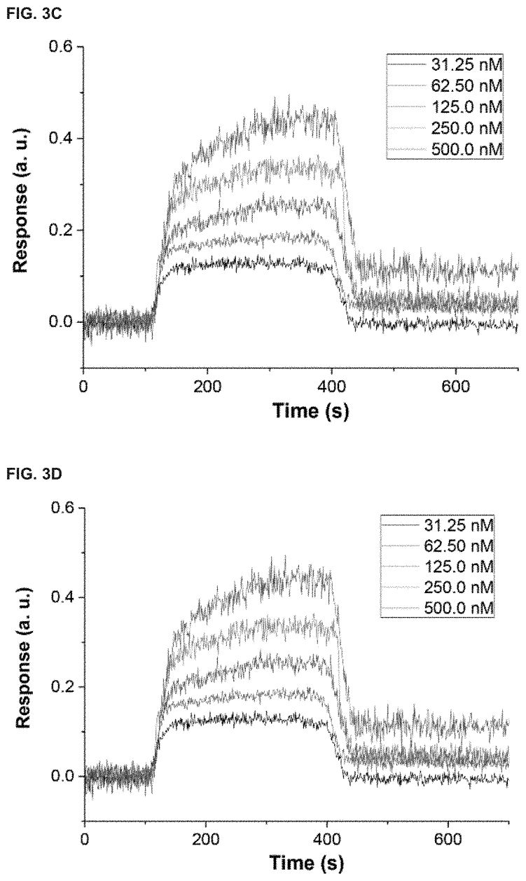

Surface Plasmon Resonance (SPR) was used to determine the binding affinity of two of the most active compounds, Compounds 1 and 2, to SARS-CoV-2 in the pseudovirus assay. SARS-CoV-2 spike (trimer) was selected in a pre-fusion state as it was hypothesized that these compounds bind to the trimer and prevent fusion of the virus to cells. Binding of these compounds to the SARS-CoV-2 spike S1 subunits containing the receptor-binding domain (RBD), which binds to the angiotensin-converting enzyme 2 (ACE2) receptor on the host cell, was further tested. This method is useful in measuring the binding constant (K_{D}) as well as kₒₙ (also known as association constant, ka) and k_{off} (also known as dissociation constant, k_{d}). Small-molecule compounds were passed through the chip surface, and the signal changes (in AU [arbitrary units]) of each compound at varied concentrations were recorded (FIG. 3A-D). The resulting data were fit to a 1:1 binding model. The binding affinity K_{D} and kinetic parameters kₒₙ and k_{off} of the target proteins' interaction with Compound 1 and Compound 2 were determined (FIG. 3E). The K_{D} value of Compound 1 and Compound 2 were 1.56 and 5.37 μM, respectively, with SARS-CoV-2 spike trimer. However, when these compounds were tested against the SARS-CoV-2 S1 subunit, the K_{D} value of Compound 1 was about 5-fold higher than when bound to the trimer. Similarly, in the case of Compound 2, the K_{D} value was about 9-fold higher when compared to the K_{D} value against the SARS-CoV-2 trimer. The data support the model that these compounds bind to the S2 subunit of the SARS-CoV-2 trimer.

TABLE 17

| Ligand | Target Protein | K_{d} | kₐ | k_{d} |
|---|---|---|---|---|
| Compound 1 | SARS-CoV-2 spike | 1.56 × 10⁻⁶ M | 1.04 × 10³ M⁻¹s⁻¹ | 1.62 × 10-³s⁻¹ |
| | (active trimer) His-tag | | | |
| Compound 2 | SARS-CoV-2 spike | 5.37 × 10⁻⁶ M | 8.72 × 10² M⁻¹s⁻¹ | 4.68 × 10-³s⁻¹ |
| | (active trimer) His-tag | | | |
| Compound 1 | SARS-CoV-2 spike S1 | 8.37 × 10⁻⁵ M | 3.07 × 10² M⁻¹s⁻¹ | 2.57 × 10-²s⁻¹ |
| | subunit protein | | | |
| Compound 2 | SARS-CoV-2 spike S1 | 5.78 × 10⁻⁵ M | 4.34 × 10² M⁻¹s⁻¹ | 2.51 × 10-²s⁻¹ |
| | subunit protein | | | |

In Vitro ADME Assessment

The in vitro assessment of ADME properties in the early stage of drug discovery and development, especially for the pharmaceutical industry, significantly reduced the drug attrition rate in the last two decades.

One of the compounds, Compound 1, with potent antiviral activity, low cytotoxicity, and excellent selectivity index (SI), was selected for evaluating its ADME properties. Permeability plays a vital role in drug absorption in the intestine and its bioavailability. Compounds with low permeability may absorb less and show poor bioavailability. The human epithelial cell line Caco-2 is the most widely used cell line to measure permeability and simulates human intestinal absorption. Therefore, the Caco-2 bidirectional permeability experiment [apical to basolateral (A-B) and basolateral to apical (B-A) across the Caco-2 cell monolayer] was performed, which can be used to measure the efflux ratio and predict the human intestinal permeability of orally administered drugs. The data shown in Table 18 indicates that the apparent permeabilities of Compound 1 are similar to the oral drug propranolol, Papp, 10⁻⁶ cm/s of which was 19.7. Valspodar, a P-gp substrate, was used as a positive control to determine whether there was any involvement of active efflux mediated by P-gp. The efflux ratio after treatment with 1 μM valspodar compared to no valspodar did not change, indicating that the P-gp mediated efflux was not involved.

TABLE 18

| Assay performed | | in vitro ADMET | Compound 1 |
|---|---|---|---|
| Solubility (μM) | | Phosphate buffer, pH 7.4 | 4.72 |
| Caco-2 | Compound 1 | A-to-B | 16.9 |
| permeability | | B-to-A | 20.4 |
| (mean Pₐₚₚ, | | Efflux Ratio | 1.21 |
| × 10⁻⁶ | Compound 1 + | A-to-B | 20.5 |
| cm/sec) | 1 μM | B-to-A | 23.6 |
| | valspodar | Efflux Ration | 1.15 |
| | P-gp Substrate | — | Negative |
| | classification | | |

| | | |
|---|---|---|
| Metabolic Stability (human | CIᵢₙₜ (mL/min/mg protein) | 0.0124 |
| liver microsomes) | Half-life (min) | 112 |
| Protein binding (human | % bound | >99.5 |
| plasma) | | |
| Cytochrome P450 inhibition, | CYP1A2 (Phenacetin) | 7.40 |
| IC₅₀ (μM) | CYP2B6 (Bupropion) | 3.19 |
| | CYP2C8 (Amodiaquine) | 2.08 |
| | CYP2C9 (Diclofenac) | 5.01 |
| | CYP2C19 (S- | 7.31 |
| | Mephenytoin) | |
| | CYP2D6 (Bufuralol) | >10 |
| | CYP3A (Midazolam) | >10 |
| | CYP3A (Testosterone) | >10 |

The metabolic stability of Compound 1 was then examined in human liver microsomes because the liver is the most crucial site of drug metabolism in the body. The clearance data (Clᵢₙₜ) in Table 18 indicated that Compound 1 is a low-clearance compound, and it shows a half-life of 112 min. It is worthwhile to mention that achieving low clearance is often the goal of a drug discovery candidate to reduce drug dose, minimize exposure of the drugs in the body, and prolong half-life. Compounds with high clearance values are cleared rapidly from the body, and the drugs have a short duration of action and may need multiple dosing. The binding of Compound 1 in human plasma was also measured, and Table 18 shows that the compound is >99.5% bound. Although it appears that Compound 1 has high protein binding, many drugs have >98% plasma protein binding, and it does not affect the success of any drug candidates.

The cytochrome P450 (CYP450) enzyme family plays a critical role in the oxidative biotransformation of many drugs and other lipophilic xenobiotics into hydrophilic counterparts, facilitating their elimination from the body. There are more than 50 CYP450 enzymes in the family, but about a dozen of them, e.g., CYP1A2, CYP2B6, CYP2C8, CYP2C9, CYP2C19, CYP2D6, CYP3A4, and CYP3A5, play an essential role in metabolizing almost 80 percent of all drugs. Therefore, a set of eight CYP450 enzymes was used to determine whether Compound 1 has any inhibitory effects on this subfamily of enzymes that may cause potential drug-drug interactions (DDI) when co-administered with other treatment agents.

The following guideline is used for the CYP inhibition assessment:
IC₅₀ >10 μM (CYP inhibition low)
<10 μM (CYP inhibition moderate)
<3 μM (CYP inhibition high)

Based on the above classification, Compound 1 showed low inhibition against CYP2D6, CYP3A, moderate inhibition against CYP1A2, CYP2B6, CYP2C9, and CYP2C19, and high inhibition against only CYP2C8 enzyme (Table 18). However, it is worth noting that Walsky et al. reported the inhibition of 209 drugs, and they classified high inhibition when IC₅₀<1 μM and ICₛₒ >10 μM as moderate inhibition. This group listed felodipine, a hypertensive drug, along with five others as highly potent inhibitors (Walsky et al. J Clin Pharmacol, 45(1):68-78, 2005).

In vivo Pharmacokinetics (PK) of Compound 1 and Compound 2

Successful drug discovery depends not only on the preclinical efficacy and toxicity profile of a compound but also on the selection of the right candidate with good in vivo pharmacokinetics in animals (e.g., rat, dog) using appropriate dosing routes, such as oral (PO) and intravenous (IV).

Evaluation of the PK parameters of two of the most active compounds was carried out in rats (Table 19) by PO and IV routes. The half-life (t_{1/2}) by PO of Compound 1 was 11.3 hours, and IV was 3.57 hours. Compound 1 dosed via IV showed Tₘₐₓ at 0.25 hours and PO at 2 hours, suggesting normal Clearance. The Cₘₐₓ, which measures the highest drug concentration in the blood or target organ for Compound 1 and Compound 2, was 1499 ng/mL and 2219 ng/mL, respectively. Compound 1 also showed an excellent mean residence time (MRT) of 14 hours. MRT measures the average time a drug molecule spends in the body and is critically important for a drug to elicit its action. The oral bioavailability of Compound 1 was reasonably good (F%: 20) for initiating further pre-clinical studies. Its bioavailability can be further improved through proper dosing, salt formation, or proper clinical formulation. Compound 2 showed poor oral availability at 0.9% and half-life by PO and IV at 3.5 to 3.9 hours. Compound 2 dosed via IV showed Cₘₐₓ is at 2 hours, suggesting compound potentially precipitated after injection and redissolved to delay maximum blood levels. The PK studies can be further evaluated by lowering the dose (1-3 mg/Kg body weight).

| | Compound 1 | | Compound 2 | | |
|---|---|---|---|---|---|
| Parameters^{a} | PO | IV | PO | IV | Units |
| Dose | 10 | 5 | 10 | 5 | mg/kg |
| t _{1/2} | 11.32 | 3.57 | 3.96 | 3.52 | hr |
| Tₘₐₓ | 2 | 0.25 | 4 | 2 | hr |
| Cₘₐₓ | 1499.76 | 7815.76 | 30.12 | 2219.45 | ng/mL |
| C₀ | — | 7407.25 | — | 1710.87 | ng/mL |
| AUC₀₋₁ | 12023.00 | 37515.91 | 318.78 | 17074.45 | ng/mL*hr |
| MRT_{0-inf}_obs | 14.34 | 4.02 | 7.28 | 5.09 | hr |
| CL_{obs} | — | 0.00013 | — | 0.00027 | mg/kg/(ng/mL)/hr |
| Vss__{obs} | — | 0.00053 | 0 | 0.00140 | mg/kg/(ng/mL) |
| Vz/F__{obs} | 0.01078 | — | 0.17593 | — | mg/kg/(ng/mL) |
| CL/F__{obs} | 0.00066 | — | 0.03082 | — | mg/kg/(ng/mL)/h |
| F % | 20.1 | — | 0.9 | — | 100*AUC(PO)/AUC(IV) |

^{a}A single oral (PO) or IV dose;
t_{1/2}, apparent terminal elimination half-life;
Tₘₐₓ, time to peak concentration;
Cₘₐₓ, maximum measured plasma concentration;
C₀, initial measured plasma concentration;
AUC, area under the concentration time curve;
MRT, mean residence time;
CL, clearance rate of the analyte (IV only);
VZ/F, apparent volume of distribution;
CL/F, apparent oral clearance;
F %, bioavailability, represents the fraction of a dose reaching systemic circulation intact, i.e. fraction of dose absorbed.

Overall, a series of pancoronavirus fusion inhibitors presented potent inhibition against the COVID-19 variants recently identified in the UK (Alpha), South Africa (Beta), and India (Delta). At least three of them (e.g., Compounds 1, 2, and 18) showed low nM IC₅₀ in a pseudovirus-based inhibition assay. These molecules also showed complete inhibition of CPE (IC₁₀₀) against an authentic live virus, SARS-CoV-2 (strain US_WA-1/2020), tested in Vero cells. Although limited, the SAR indicates that a balance of electrostatic and hydrophobic interactions is needed for optimum antiviral activity. For example, when phenylethyl moiety was replaced by H or smaller hydrophobic groups, the inhibitory activity of those compounds disappeared. The SAR also shows that there is room for further derivatization of the phenylethyl moiety. The direct binding study by SPR confirmed that these molecules bind to the prefusion trimer of the spike protein of SARS-CoV-2 more tightly than the 51 subdomain of the spike protein. Subsequent cell-to-cell fusion assay confirmed that these compounds efficiently prevent virus-mediated cell-to-cell fusion. The compounds were not promiscuous but true pancoronavirus inhibitors despite the presence of ene-rhodanine scaffold, which was termed by some as “frequent hitters”. The ADME properties measured showed drug-like characteristics of the compounds. Furthermore, the pharmacokinetic (PK) study in rats demonstrated that Compound 1 has all the desirable features, including 20% oral availability to be considered for further pre-clinical assessments.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, such as within 5 to 10%. Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of the present disclosure are described herein, including the best mode known to the inventors for carrying out the present disclosure. Of course, variations on these described embodiments will become apparent to those of ordinary skill in the art upon reading the foregoing description. The inventor expects skilled artisans to employ such variations as appropriate, and the inventors intend for the present disclosure to be practiced otherwise than specifically described herein. Accordingly, the present disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the present disclosure disclosed herein are illustrative of the principles of the present disclosure. Other modifications that may be employed are within the scope of the present disclosure. Thus, by way of example, but not of limitation, alternative configurations of the present disclosure may be utilized in accordance with the teachings herein. Accordingly, the present disclosure is not limited to that precisely as shown and described.

1. A method of treating a coronavirus infection comprising administering a compound having a structure represented by Formula (I) : 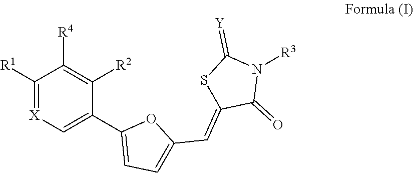
or a pharmaceutically acceptable salt thereof,
wherein
X is CH or N;
Y is S, O, or NH;
R¹ is H, halogen or C₁₋₆ alkyl;
R² is H, halogen or C₁₋₆ alkyl;
R³ is H, methyl, ethyl, isobutyl, methoxyethyl, phenylethyl, allyl, propynyl, cyclohexyl, C₁₋₆ alkyl, C₁₋₆ alkoxyalkyl, C₁₋₆ alkenyl, or C₁₋₆ alkynyl; and
R⁴ is H or COOH.

2. The method of claim 1, wherein the compound has the structure of Formula (II): 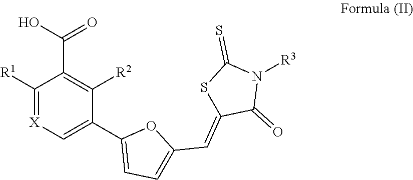
or a pharmaceutically acceptable salt thereof,
wherein
X is CH or N;
R¹ is H, halogen or C₁₋₆ alkyl;
R² is H, halogen or C₁₋₆ alkyl; and
R³ is H, C₁₋₆ alkyl, C₁₋₆ alkoxyalkyl, C₁₋₆ alkenyl, C₁₋₆ alkynyl, C₃₋₆ cycloalkyl, -(C₁₋₄ alkyl)-phenyl.

3. The method of claim 1, wherein the compound has a structure of Formula (III): 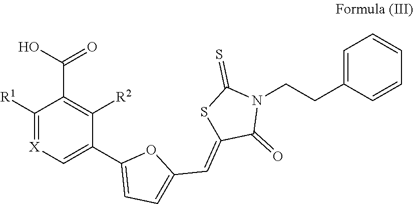
or a pharmaceutically acceptable salt thereof,
wherein X is CH or N;
R¹ is H, halogen or C₁₋₆ alkyl; and
R² is H, halogen or C₁₋₆ alkyl.

4. (Withdrawn and Amended) The method of claim 1, wherein the compound has a structure of Formula (IV): 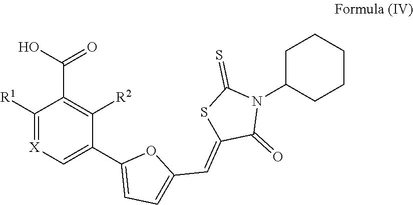
or a pharmaceutically acceptable salt thereof
wherein X is CH or N;
R¹ is H, halogen or C₁₋₆ alkyl; and
R² is H, halogen or C₁₋₆ alkyl.

5. The method of claim 1, wherein R² is H.

6. The method of claim 1, wherein R¹ is methyl, Cl, or Br.

7. The method of claim 1, wherein R¹ is H.

8. The method of claim 1, wherein R³ is phenylethyl or cyclohexyl.

9. The method of claim 1, wherein the compound is selected from the compounds of Table 1.

10. The method of claim 1, wherein the compound is

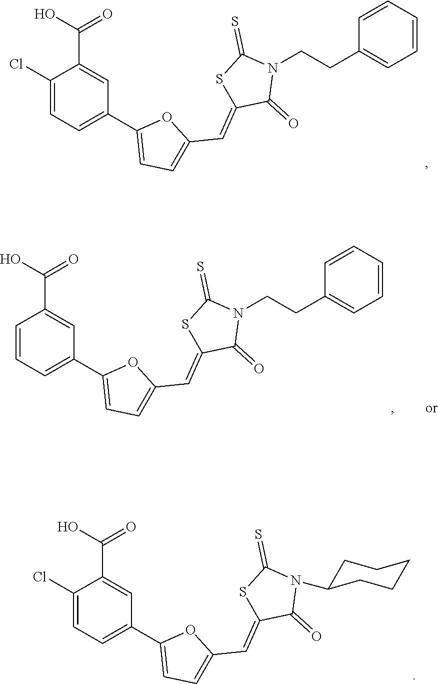 [SMILES: O=C(O)c1cc(-c2ccc(/C=C3\SC(=S)N(CCc4ccccc4)C3=O)o2)ccc1Cl ; O=C(O)c1cccc(-c2ccc(/C=C3\SC(=S)N(CCc4ccccc4)C3=O)o2)c1 ; O=C(O)c1cc(-c2ccc(/C=C3\SC(=S)N(C4CCCCC4)C3=O)o2)ccc1Cl]

11. The method of claim 1, wherein the subject in need thereof has the coronavirus infection confirmed by detection of a coronavirus virus in a biological sample from the subject.

12. (canceled)

13. The method of claim 1, wherein the coronavirus is SARS-CoV-2.

14. The method of claim 1, wherein the compound is administered orally.

15. The method of claim 1, wherein the compound is administered intranasally.

16-29. (canceled)


SEQUENCE LISTING (10 sequences)

SEQ ID NO: 1 - DNA, 42 bp, Artificial Sequence
  SaCoV2-E484K-F primer
accccttgtaacggcgtgaaaggcttcaactgctacttccca

SEQ ID NO: 2 - DNA, 41 bp, Artificial Sequence
  SaCoV2-E484K-REV primer
tgggaagtagcagttgaagcctttcacgccgttacaagggg

SEQ ID NO: 3 - DNA, 42 bp, Artificial Sequence
  SaCoV2-N501Y-F primer
tcctacggctttcagcccacatatggcgtgggctatcagccc

SEQ ID NO: 4 - DNA, 42 bp, Artificial Sequence
  SaCoV2-N501Y-REV primer
gggctgatagcccacgccatatgtgggctgaaagccgtagga

SEQ ID NO: 5 - DNA, 42 bp, Artificial Sequence
  SaCoV2-D614G-F primer
caggtggcagtgctgtaccagggcgtgaactgtaccgaagtg

SEQ ID NO: 6 - DNA, 42 bp, Artificial Sequence
  SaCoV2-D614G-REV primer
cacttcggtacagttcacgccctggtacagcactgccacctg

SEQ ID NO: 7 - DNA, 39 bp, Artificial Sequence
  SaCoV2-P681H-F primer
cagacacagacaaacagccacagacgggccagatctgtg

SEQ ID NO: 8 - DNA, 39 bp, Artificial Sequence
  SaCoV2-P681H-REV primer
cacagatctggcccgtctgtggctgtttgtctgtgtctg

SEQ ID NO: 9 - DNA, 41 bp, Artificial Sequence
  SaCoV2-?(69-70)-S primer
gtgacctggttccacgccatctccggcaccaatggcaccag

SEQ ID NO: 10 - DNA, 42 bp, Artificial Sequence
  SaCoV2-?(69-70)-REV primer
cttggtgccattggtgccggagatggcgtggaaccaggtcac